(19) United States
(12) Patent Application Publication
(10) Pub. No.: US 2007/0032522 A1
(43) Pub. Date: Feb. 8, 2007

(54) Antiviral agents

(76) Inventors: Dange Vijay Kumar, Salt Lake City, UT (US); Roopa Rai, San Carlos, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(21) Appl. No.: 11/478,371

(22) Filed: Jun. 28, 2006

Related U.S. Application Data
(60) Provisional application No. 60/696,063, filed on Jul. 1, 2005.

(51) Int. Cl. A61K 31/4704

(52) U.S. Cl. 514/312; 546/156

(57) ABSTRACT

The present invention is directed to compounds that are antiviral agents. Specifically the compounds of the present invention inhibit replication of HCV and are therefore useful in treating hepatitis C infections. The present invention is also directed to pharmaceutical compositions comprising these compounds and processes for preparing them.

### CROSS-REFERENCES TO RELATED APPLICATIONS

This application claims the benefit of U.S. Provisional application Ser. No. 60/696,063, filed Jul. 1, 2005, the disclosure of which is incorporated herein by reference.

### STATEMENT AS TO RIGHTS TO INVENTIONS MADE UNDER FEDERALLY SPONSORED RESEARCH OR DEVELOPMENT

NOT APPLICABLE

### REFERENCE TO A “SEQUENCE LISTING,” A TABLE, OR A COMPUTER PROGRAM LISTING APPENDIX SUBMITTED ON A COMPACT DISK.

NOT APPLICABLE

### FIELD OF THE INVENTION

The present invention is directed to compounds that are antiviral agents. Specifically the compounds of the present invention inhibit replication of HCV and are therefore useful in treating hepatitis C infections. The present invention is also directed to pharmaceutical compositions comprising these compounds and processes for preparing them.

### BACKGROUND OF THE INVENTION

### State of the Art

Hepatitis C virus (HCV) is a (+)-sense single-standed RNA virus that is a major cause of non-A, non-B hepatitis worldwide. A large percentage of people infected with HCV develop chronic liver disease, the so called chronic hepatitis C which in turn makes them at high risk for developing serious liver diseas such as liver cirrhosis, hepatocellular carcinoma and terminal liver disease leading to death. Currently, hepatitits C is treated with either interferon alone or in combination with Ribavirin or with pegylated forms of interferon such as PEG-Intron® and Pegasys®. These therapies, however, induce severe side effects such as retinopathy, thyroiditis, acute pancreatitis, depression. Therefore, there is a need for safe, oral drug for the treatment of hepatitis C. The present invention fulfils this and related needs.

### BRIEF SUMMARY OF THE INVENTION

In one aspect, this invention is directed to a compound of Formula (I): 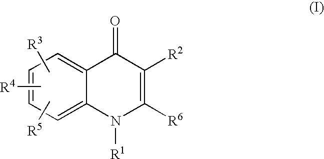
where:

R¹ is -(alkylene)-R⁷ where the alkylene chain is optionally substituted with one or two halo and R⁷ is cycloalkyl, aryl, heteroaryl, or heterocycloalkyl wherein the aromatic and alicyclic rings are optionally substituted with one, two, or three R^{a} independently selected from alkyl, alkoxy, hydroxyl, halo, haloalkyl, haloalkoxy, alkylsulfonyl, hydroxyalkyl, alkoxyalkyl, aminoalkyl, alkylamino, hydroxyalkoxy, alkoxyalkyloxy, aminoalkoxy, optionally substituted phenyloxy, optionally substituted phenalkyloxy, optionally substituted heteroaryloxy, optionally substituted heteroaralkyloxy, optionally substituted heterocycloalkyloxy, optionally substituted heterocycloalkylalkyloxy, carboxy, carboxyalkyl, alkoxycarbonyl, acyl, acylamino, acylaminoalkyl, acyloxy, aminocarbonyl, aminocarbonylalkyl, aminosulfonyl, aminosulfonylalkyl, sulfonylamino, sulfonylaminoalkyl, cyano, ureido, ureidoalkyl, aminocarbonyloxy, aminocarbonylalkyloxy, or alkoxycarbonylamino;

R² is —COR⁸ (where R⁸ is cycloalkyl, cycloalkylalkyl, aryl, aralkyl, heteroaryl, or heteroaralkyl), —COOR⁹ (where R⁹ is alkyl, cycloalkyl, cycloalkylalkyl, aryl, aralkyl, heteroaryl, or heteroaralkyl), —CONR¹⁰R¹¹ (where R¹⁰ hydrogen, alkyl, aryl, aralkyl, heteroaryl, or heteroaralkyl and R¹¹ is alkyl, cycloalkyl, cycloalkylalkyl, heterocycloalkylalkyl, aryl, aralkyl, heteroaryl, or heteroaralkyl or R¹⁰ and R¹¹ together with the nitrogen atom to which they are attached form heterocycloamino), —NR¹²R¹³ (where R¹² is hydrogen, alkyl, hydroxyalkyl, alkoxyalkyl, aryl, aralkyl, heteroaryl, heteroaralkyl, or heterocycloalkylalkyl and R¹³ is hydrogen, alkyl, cycloalkyl, cycloalkylalkyl, heterocycloalkylalkyl, aryl, aralkyl, heteroaryl, heteroaralkyl, or heterocycloalkylalkyl), —C(OH)R¹⁴R⁸ (where R¹⁴ is hydrogen, alkyl, aryl, aralkyl, heteroaryl, or heteroaralkyl and R⁸ is aryl, aralkyl, heteroaryl, or heteroaralkyl), —SO₂NR¹⁶R¹⁷ (where R¹⁶ hydrogen, alkyl, aryl, aralkyl, heteroaryl, or heteroaralkyl and R¹⁷ is alkyl, aryl, aralkyl, heteroaryl, or heteroaralkyl or R¹⁶ and R¹⁷ together with the nitrogen atom to which they are attached form heterocycloamino), —NHSO₂R¹⁸ (where R¹⁸ is aryl, aralkyl, heteroaryl, heteroaralkyl, or heterocycloalkyl), —NHCOOR¹⁹ (where R¹⁹ is aryl, aralkyl, heteroaryl, heteroaralkyl, or heterocycloalkyl), —NHCONHR²⁰R²¹ (where R²⁰ is hydrogen or alkyl and R²¹ is aryl, aralkyl, heteroaryl, heteroaralkyl, or heterocycloalkyl or R²⁰ and R²¹ together with the nitrogen atom to which they are attached form heterocycloamino), —SR²², —SOR²² (where in each instance R²² is aryl, aralkyl, heteroaryl, or heteroaralkyl); —SO₂R²³ (where R²³ is aryl, aralkyl, heteroaryl, or heteroaralkyl), or —SO₃R²⁴ where R²⁴ is aryl, aralkyl, heteroaryl, or heteroaralkyl); wherein the aromatic, alicyclic or heterocycloalkyl ring in R² is optionally substituted with one, two, or three R^{b} independently selected from alkyl, alkoxy, hydroxyl, halo, haloalkyl, haloalkoxy, alkylsulfonyl, hydroxyalkyl, alkoxyalkyl, aminoalkyl, alkylamino, hydroxyalkoxy, alkoxyalkyloxy, aminoalkoxy, optionally substituted phenyloxy, optionally substituted phenylalkyloxy, optionally substituted heteroaralkyloxy, optionally substituted heterocycloalkyloxy, carboxy, carboxyalkyl, alkoxycarbonyl, alkoxycarbonylalkyl, acyl, acylamino, acylaminoalkyl, acyloxy, aminocarbonyl, aminocarbonylalkyl, aminosulfonyl, aminosulfonylalkyl, sulfonylamino, sulfonylaminoalkyl, cyano, ureido, ureidoalkyl, aminocarbonyloxy, or alkoxycarbonylamino;

R³ is hydrogen, alkyl, halo, alkoxy, hydroxy, haloalkoxy, or haloalkyl;

R⁴ is hydrogen, alkyl, halo, alkoxy, hydroxy, haloalkoxy, haloalkyl, alkylthio, alkylsulfonyl, hydroxyalkyl, alkoxyalkyl, aminoalkyl, hydroxyalkoxy, alkoxyalkyloxy, aminoalkoxy, aryloxy, aralkyloxy, heteroaralkyloxy, heterocycloalkyloxy, heterocycloalkylalkyloxy, carboxyalkyl, alkoxycarbonylalkyl, carboxy, alkoxycarbonyl, acyl, acylamino, acyloxy, aminocarbonyl, aminocarbonylalkyloxy, aminosulfonyl, ureido, aminocarbonyloxy, or alkoxycarbonylamino wherein the aromatic or alicyclic ring in aryloxy, aralkyloxy, heteroaralkyloxy, heterocycloalkyloxy, and heterocycloalkylalkyloxy is optionally substituted with one, two, or three R^{c} independently selected from alkyl, alkoxy, hydroxyl, halo, haloalkyl, haloalkoxy, alkylsulfonyl, hydroxyalkyl, alkoxyalkyl, aminoalkyl, hydroxyalkoxy, alkoxyalkyloxy, aminoalkoxy, optionally substituted phenyloxy, optionally substituted phenylalkyloxy, optionally substituted heteroaryloxy, optionally substituted heteroaralkyloxy, optionally substituted heterocycloalkyloxy, optionally substituted heterocycloalkylalkyloxy, carboxy, carboxyalkyl, alkoxycarbonyl, acyl, acylamino, acylaminoalkyl, acyloxy, aminocarbonyl, aminocarbonylalkyl, aminosulfonyl, aminosulfonylalkyl, sulfonylamino, sulfonylaminoalkyl, cyano, ureido, ureidoalkyl, aminocarbonyloxy, aminocarbonylalkyloxy, or alkoxycarbonylamino; and

R⁵ is hydrogen, alkyl, halo, alkoxy, hydroxy, haloalkoxy, haloalkyl, aryl, aralkyl, heteroaryl, heteroaralkyl, cycloalkyl, heterocycloalkyl, heterocycloalkylalkyl, heterocycloalkylalkyloxy, monosubstituted amino, or disubstituted amino or when R⁵ together with R³ or R⁴ forms methylenedioxy or ethylenedioxy wherein the aromatic and alicyclic ring in aryl, aralkyl, heteroaryl, heteroaralkyl, heterocycloalkyl, heterocycloalkylalkyl, and heterocycloalkylalkyloxy is optionally substituted with one, two, or three R^{d} independently selected from alkyl, alkoxy, hydroxyl, halo, haloalkyl, haloalkoxy, alkylsulfonyl, hydroxyalkyl, alkoxyalkyl, aminoalkyl, hydroxyalkoxy, alkoxyalkyloxy, aminoalkoxy, optionally substituted phenyloxy, optionally substituted phenylalkyloxy, optionally substituted heteroaryloxy, optionally substituted heteroaralkyloxy, optionally substituted heterocycloalkyloxy, optionally substituted heterocycloalkylalkyloxy, carboxy, carboxyalkyl, alkoxycarbonyl, acyl, acylamino, acylaminoalkyl, acyloxy, aminocarbonyl, aminocarbonylalkyl, aminosulfonyl, aminosulfonylalkyl, sulfonylamino, sulfonylaminoalkyl, cyano, ureido, ureidoalkyl, aminocarbonyloxy, aminocarbonylalkyloxy, or alkoxycarbonylamino; and

R⁶ is hydrogen, alkyl, halo, alkoxy, alkylamino, dialkylamino, phenyl, heteroaryl, carboxy, alkoxycarbonyl, or optionally substituted heterocycloalkylalkyloxycarbonyl; or

a pharmaceutically acceptable salts thereof, provided that the compound of Formula (I) is not:

L-proline, 1-[[1,4-dihydro-4-oxo-1-(phenylmethyl)-3-quinolinyl]carbonyl] ethyl ester;

3-benzoyl-6-methyl-1-(phenylmethyl)-4(1H)quinolinone;

6-methoxy-3-(4-methoxybenzoyl)-1-(phenylmethyl)-4(1H)quinolinone;

3-benzoyl-5,7-dimethoxy-1-(phenylmethyl)-4(1H)quinolinone;

3-(4-methoxybenzoyl)-6-methyl-1-(phenylmethyl)-4(1H)quinolinone;

3-(1,3-benzodioxol-5-ylcarbonyl)-1-(phenylmethyl)-4(1H)quinolinone;

1-[(1,4-dihydro-4-oxo-1-phenethyl-3-quinolyl)carbonyl]piperidine;

1-[(1,4-dihydro-4-oxo-1-phenethyl-3-quinolyl)carbonyl]-4-methylpiperazine;

1-[(1,4-dihydro-4-oxo-1-phenethyl-3-quinolyl)carbonyl]morpholine; and

when R¹ is benzyl optionally substituted with methyl, chloro, methoxy, or fluoro, R² is —COR⁸ where R⁸ is phenyl optionally substituted with methyl, ethyl, methoxy, ethoxy, fluoro, or chloro or phenyl disubstituted with methyl, R³ is hydrogen, and R⁴ is methyl, methoxy, ethoxy, or fluoro, then R⁵ is not hydrogen, methoxy, or fluoro.

In a second aspect, this invention is directed to a pharmaceutical composition comprising a compound of Formula (I) or a pharmaceutically acceptable salt thereof in admixture with one or more suitable excipients.

In a third aspect, this invention is directed to a method for treating hepatitis C in an animal which method comprises administering to the animal a pharmaceutical composition comprising a therapeutically effective amount of a compound of Formula (I) or a pharmaceutically acceptable salt thereof in admixture with one or more suitable excipients.

In a fourth aspect, this invention is directed to processes for preparing compounds of Formula (I).

### BRIEF DESCRIPTION OF THE DRAWINGS

NOT APPLICABLE

### DETAILED DESCRIPTION OF THE INVENTION

### Definitions

Unless otherwise stated, the following terms used in the specification and claims are defined for the purposes of this Application and have the following meanings.

“Alicyclic” means a moiety characterized by arrangement of the carbon atoms in closed non aromatic ring structures e.g., cycloalkyl and heterocycloalkyl rings as defined herein.

“Alkyl” represented by itself means a straight or branched, saturated aliphatic radical containing one to eight carbon atoms, unless otherwise indicated e.g., alkyl includes methyl, ethyl, propyl, isopropyl, butyl, sec-butyl, isobutyl, tert-butyl, and the like.

“Alkylene”, unless indicated otherwise, means a straight or branched, saturated aliphatic, divalent radical having the number of one to six carbon atoms, e.g., methylene (—CH₂—), ethylene (—CH₂CH₂—), trimethylene (—CH₂CH₂CH₂—), tetramethylene (—CH₂CH₂CH₂CH₂—) 2-methyltetramethylene (—CH₂CH(CH₃)CH₂CH₂—), pentamethylene (—CH₂CH₂CH₂CH₂CH₂—), and the like.

“Alkylthio” means an —SR radical where R is alkyl as defined herein, e.g., methylthio, ethylthio, propylthio, or butylthio, and the like.

“Alkylsulfonyl” means —SO₂R radical where R is alkyl as defined herein e.g., methylsulfonyl, ethylsulfonyl, and the like.

“Alkoxy” refers to a —OR radical where R is an alkyl group as defined above e.g., methoxy, ethoxy, and the like.

“Alkoxycarbonylamino” refers to a —NHC(O)OR radical where R is an alkyl group as defined above e.g., methoxycarbonylamino, ethoxycarbonylamino, and the like.

“Alkoxyalkyl” means a linear monovalent hydrocarbon radical of one to six carbon atoms or a branched monovalent hydrocarbon radical of three to six carbons substituted with at least one alkoxy group, preferably one or two alkoxy groups, as defined above, e.g., 2-methoxy-ethyl, 1-, 2-, or 3-methoxypropyl, 2-ethoxyethyl, and the like.

“Alkoxyalkyloxy” refers to a —OR radical where R is alkoxyalkyl as defined above e.g., 2-methoxyyethoxy, 2,3-dimethoxypropoxy, and the like.

“Alkoxycarbonyl” refers to a —C(O)OR radical where R is an alkyl group as defined above e.g., methoxycarbonyl, ethoxycarbonyl, and the like.

“Alkoxycarbonylalkyl” refers to a -(alkylene)R radical where R is an alkoxycarbonyl group as defined above e.g., methoxycarbonylethyl, ethyoxycarbonylethyl, and the like.

“Amino” means a —NH₂ radical.

“Alkylamino” means a radical —NHR where R is alkyl as defined herein, e.g., methylamino, ethylamino, n-, iso-propylamino, n-, iso-, tert-butylamino, and the like.

“Aminoalkyl” means a linear monovalent hydrocarbon radical of one to six carbon atoms or a branched monovalent hydrocarbon radical of three to six carbons substituted with at least one, preferably one or two, —NRR′ where R is hydrogen, alkyl, acyl, hydroxyalkyl, alkoxyalkyl, optionally substituted phenyl, optionally substituted phenalkyl, optionally substituted heteroaryl, optionally substituted heteroaralkyl or optionally substituted heterocycloalkylalkyl and R′ is hydrogen, alkyl, hydroxyalkyl, alkoxyalkyl, optionally substituted phenyl, optionally substituted phenalkyl, optionally substituted heteroaryl, optionally substituted heteroaralkyl or optionally substituted heterocycloalkylalkyl, cycloalkyl, cycloalkylalkyl, aminocarbonyl, or aminosulfonyl as defined herein e.g., aminomethyl, methylaminoethyl, dimethylaminoethyl, 1,3-diaminopropyl, acetylaminopropyl, and the like.

“Aminoalkoxy” refers to a —OR radical where R is aminoalkyl as defined above e.g., 2-methylaminoethoxy, 3-dimethylaminopropoxy, 2,3-dimethylaminopropoxy, and the like.

“Acyl” refers to a —COR radical where R is hydrogen, alkyl, haloalkyl, cycloalkyl, optionally substituted phenyl, optionally substituted phenalkyl, optionally substituted heteroaryl, optionally substituted heteroaralkyl, optionally substituted heterocycloalkyl, or optionally substituted heterocycloalkylalkyl as defined herein, e.g., formyl, acetyl, trifluoroacetyl, benzoyl, piperazin-1-ylcarbonyl, and the like. When R is alkyl it is referred to in this application as alkylcarbonyl. When R is aryl it is referred to in this application as optionally substituted phenylcarbonyl. When R is optionally substituted heteroaryl it is referred to in this application as optionally substituted heteroarylcarbonyl. When R is optionally substituted heterocycloalkyl it is referred to in this application as optionally substituted heterocycloalkylcarbonyl.

“Acyloxy” refers to a —OR radical where R is acyl group as defined above e.g., acetyloxy, propionyloxy, benzoyloxy, and the like.

“Acylamino” refers to a —NRCOR′ radical where R is hydrogen or alkyl and R′ is hydrogen, alkyl, haloalkyl, cycloalkyl, optionally substituted phenyl, optionally substituted phenalkyl, optionally substituted heteroaryl, optionally substituted heteroaralkyl or optionally substituted heterocycloalkylalkyl as defined herein, e.g., formyl, acetyl, trifluoroacetyl, benzoyl, piperazin-1-ylcarbonyl, and the like.

“Acylaminoalkyl” refers to a -(alkylene)R radical where R is an acylamino group as defined above e.g., acetylaminoethyl, and the like.

“Aminocarbonyl” means —CONRR′ radical where R and R′ are independently selected from hydrogen, alkyl, hydroxyalkyl, alkoxyalkyl, alkoxyalkyloxyalkyl, hydroxyalkyloxyalkyl, optionally substituted phenyl, optionally substituted phenalkyl, optionally substituted heteroaryl, optionally substituted heteroaralkyl or optionally substituted heterocycloalkylalkyl or R and R′ together with the nitrogen atom to which they are attached form optionally substituted heterocycloamino as defined herein.

“Aminocarbonylalkyl” refers to a -(alkylene)R radical where R is an aminocarbonyl group as defined above e.g., aminocarbonylethyl, methylaminocarbonylethyl, dimethylaminocarbonylethyl, benzylaminocarbonylethyl, piperazin-1-ylcarbonylmethyl, piperidin-1-ylcarbonylethyl, and the like.

“Aminocarbonylalkyloxy” refers to a —O-(alkylene)R radical where R is an aminocarbonylalkyl group as defined above e.g., aminocarbonylethyloxy, methylaminocarbonylethyloxy, dimethylaminocarbonylethyloxy, benzylaminocarbonylethyloxy, piperazin-1-ylcarbonylmethyloxy, piperidin-1-ylcarbonylethyloxy, and the like.

“Aminocarbonyloxy” means —OCONRR′ radical where R and R′ are independently selected from hydrogen, alkyl, optionally substituted phenyl, optionally substituted phenalkyl, optionally substituted heteroaryl, optionally substituted heteroaralkyl or optionally substituted heterocycloalkylalkyl or R and R′ together with the nitrogen atom to which they are attached form optionally substituted heterocycloamino as defined herein.

“Aminosulfonyl” means —SO₂NRR′ radical where R and R′ are independently selected from hydrogen, alkyl, optionally substituted phenyl, optionally substituted phenalkyl, optionally substituted heteroaryl, optionally substituted heteroaralkyl or optionally substituted heterocycloalkylalkyl or R and R′ together with the nitrogen atom to which they are attached form optionally substituted heterocycloamino as defined herein.

“Aminosulfonylalkyl” refers to a -(alkylene)R radical where R is an aminosulfonyl group as defined above e.g., aminosulfonylethyl, methylaminosulfonylethyl, dimethylaminosulfonylethyl, benzylaminosulfonylethyl, piperazin-1-ylsulfonylmethyl, piperidin-1-ylsulfonylethyl, and the like.

“Animal” includes humans, non human mammals (e.g., dogs, cats, rabbits, cattle, horses, sheep, goats, swine, deer, and the like) and non mammals (e.g., birds, and the like).

“Aromatic” refers to a moiety wherein the constituent atoms make up an unsaturated ring system, all atoms in the ring system are sp² hybridized and the total number of pi electrons is equal to 4n+2.

“Aryl” refers to a monocyclic or fused bicyclic ring assembly containing 6 to 10 ring carbon atoms wherein each ring is aromatic e.g., phenyl or naphthyl.

“Aryloxy” refers to a —O—R radical where R is aryl as defined above e.g., phenoxy, napthyloxy, and the like.

“Aralkyl” refers to a -(alkylene)-R radical where R is aryl as defined above e.g., benzyl, phenethyl, and the like.

“Aralkyloxy” refers to a —O—R radical where R is aralkyl as defined above e.g., benzyloxy, 2-phenethoxy, and the like.

“Carboxy” refers to —C(O)OH radical.

“Carboxyalkyl” means an alkyl radical, as defined herein, substituted with at least one, preferably one or two, —C(O)OR group(s) where R is hydrogen, alkyl, optionally substituted phenyl, optionally substituted heteroaryl, or optionally substituted heterocycloalkyl, e.g., carboxymethyl, carboxyethyl, 1-, 2-, or 3 carboxypropyl, and the like.

“Cycloalkyl” refers to a monovalent saturated monocyclic ring containing three to eight ring carbon atoms e.g., cyclopropyl, cyclobutyl, cyclopentyl, cyclohexyl, and the like.

“Cycloalkylalkyl” refers to a -(alkylene)R radical where R is an cycloalkyl group as defined above e.g., cyclopropylmethyl, cyclohexylmethyl, cyclohexylethyl, and the like.

“Disease” specifically includes any unhealthy condition of an animal or part thereof and includes an unhealthy condition that may be caused by, or incident to, medical or veterinary therapy applied to that animal, i.e., the “side effects” of such therapy.

“Dialkylamino” means a radical —NRR′ where R and R′ are independently alkyl as defined herein, e.g., dimethylamino, diethylamino, N,N-methylpropylamino or N,N-methylethylamino, and the like.

“Disubstituted amino” means a radical —NRR′ where R and R′ are independently selected from alkyl, hydroxyalkyl, alkoxyalkyl, aminoalkyl, optionally substituted phenyl, optionally substituted phenalkyl, optionally substituted heteroaryl, or optionally substituted heteroaralkyl as defined herein, e.g., dimethylamino, diethylamino, N,N-methylpropylamino or N,N-methylethylamino, methylphenylamino, and the like. Dialkylamino is a subgroup of disubstituted amino.

“Halo” refers to fluoro, chloro, bromo or iodo.

“Haloalkyl” refers to alkyl as defined above substituted by one or more, preferably one to seven, “halo” atoms, as such terms are defined in this Application. Haloalkyl includes monohaloalkyl, dihaloalkyl, trihaloalkyl, perhaloalkyl and the like e.g. chloromethyl, dichloromethyl, difluoromethyl, trifluoromethyl, 2,2,2-trifluoroethyl, perfluoroethyl, 2,2,2-trifluoro-1,1-dichloroethyl, and the like.

“Haloalkoxy” refers to a —OR radical where R is haloalkyl group as defined above e.g., trifluoromethoxy, 2,2,2-trifluoroethoxy, difluoromethoxy, and the like.

“Heteroaryl” as a group or part of a group denotes an aromatic monocyclic or bicyclic moiety of 5 to 10 ring atoms in which one or more, preferably one, two, or three, of the ring atom(s) is(are) selected from nitrogen, oxygen or sulfur, the remaining ring atoms being carbon. Representative heteroaryl rings include, but are not limited to, pyrrolyl, furanyl, thienyl, oxazolyl, isoxazolyl, thiazolyl, imidazolyl, triazolyl, tetrazolyl, pyridinyl, pyrimidinyl, pyrazinyl, pyridazinyl, indolyl, benzofuranyl, benzothiophenyl, benzimidazolyl, quinolinyl, isoquinolinyl, quinazolinyl, quinoxalinyl, pyrazolyl, and the like.

“Heteroaralkyl” refers to a -(alkylene)-R radical where R is heteroaryl as defined above e.g., pyridinylmethyl, 1- or 2-furanylethyl, imidazolylmethyl, and the like.

“Heteroaralkyloxy” refers to a —OR radical where R is heteroaralkyl as defined above e.g., pyridinylmethyloxy, 1- or 2-furanylethyloxy, imidazolylmethyloxy, and the like.

“Heterocycloalkyl” refers to a saturated or partially unsaturated, mono or bicyclic radical of 4, 5, 6, or 7 carbon ring atoms wherein one or more, preferably one, two, or three of the ring carbon atoms are replaced by a heteroatom selected from —N═, —N—, —O—, —S—, —SO—, or —S(O)₂— and further wherein one or two ring carbon atoms are optionally replaced by a keto (—CO—) group. The heterocycloalkyl ring is optionally fused to cycloalkyl, aryl or heteroaryl ring as defined herein. Representative examples include, but are not limited to, imidazolidinyl, morpholinyl, thiomorpholinyl, thiomorpholino-1-oxide, thiomorpholino-1,1-dioxide, tetrahydrofuranyl, tetrahydropyranyl, tetrahydrothiopyranyl, 1-oxo-tetrahydrothiopyranyl, 1,1-dioxotetrathio-pyranyl, indolinyl, piperazinyl, piperidyl, pyrrolidinyl, pyrrolinyl, quinuclidinyl,3,4-dihydroisoquinolinyl, dihydroindolyl, and the like.

When the heterocycloalkyl group contains at least one nitrogen ring atom it is referred to herein as “heterocycloamino” and is a subset of the heterocycloalkyl group as defined above.

“Heterocycloalkoxy” refers to a —OR radical where R is heterocycloalkyl as defined above e.g., pyrrolidinyloxy, tetrahydropyranyloxy, and the like.

“Heterocycloalkylalkyl” refers to a -(alkylene)-R radical where R is heterocycloalkyl as defined above e.g., pyrrolidinylmethyl, tetrahydrofuranylethyl, piperidinylmethyl, and the like.

“Heterocycloalkylalkoxy” refers to a —OR radical where R is heterocycloalkylalkyl as defined above e.g., pyrrolidinylmethyloxy, piperazinylethoxy, piperidinylethoxy, and the like.

“Heterocycloalkylalkoxycarbonyl” or “heterocycloalkylalkyloxycarbonyl” refers to a —COR radical where R is heterocycloalkylalkyloxy as defined above e.g., pyrrolidinylmethyloxycarbonyl, piperazinylethoxycarbonyl, piperidinylethoxycarbonyl, and the like.

“Hydroxy” means —OH radical.

“Hydroxyalkyl” means a linear monovalent hydrocarbon radical of one to six carbon atoms or a branched monovalent hydrocarbon radical of three to six carbons substituted with one or two hydroxy groups, provided that if two hydroxy groups are present they are not both on the same carbon atom. Representative examples include, but are not limited to, hydroxymethyl, 2-hydroxyethyl, 2-hydroxypropyl, 3-hydroxypropyl, 1-(hydroxymethyl)-2-methylpropyl, 2-hydroxybutyl, 3-hydroxybutyl, 4-hydroxybutyl, 2,3-dihydroxypropyl, 1-(hydroxymethyl)-2-hydroxyethyl, 2,3-dihydroxybutyl, 3,4-dihydroxybutyl and 2-(hydroxymethyl)-3-hydroxypropyl, preferably 2-hydroxyethyl, 2,3-dihydroxypropyl, and 1-(hydroxymethyl)-2-hydroxyethyl.

“Hydroxyalkoxy” refers to a —OR radical where R is hydroxyalkyl as defined above e.g., 2-hydroxyethoxy, 2,3-dihydroxypropoxy, and the like.

“Isomers” mean compounds of Formula (I) having identical molecular formulae but differ in the nature or sequence of bonding of their atoms or in the arrangement of their atoms in space. Isomers that differ in the arrangement of their atoms in space are termed “stereoisomers”. Stereoisomers that are not mirror images of one another are termed “diastereomers” and stereoisomers that are nonsuperimposable mirror images are termed “enantiomers” or sometimes “optical isomers”. A carbon atom bonded to four nonidentical substituents is termed a “chiral center”. A compound with one chiral center that has two enantiomeric forms of opposite chirality is termed a “racemic mixture”. A compound that has more than one chiral center has 2ⁿ⁻¹ enantiomeric pairs, where n is the number of chiral centers. Compounds with more than one chiral center may exist as either an individual diastereomer or as a mixture of diastereomers, termed a “diastereomeric mixture”. When one chiral center is present a stereoisomer may be characterized by the absolute configuration of that chiral center. Absolute configuration refers to the arrangement in space of the substituents attached to the chiral center. Enantiomers are characterized by the absolute configuration of their chiral centers and described by the R- and S-sequencing rules of Cahn, Ingold and Prelog. Conventions for stereochemical nomenclature, methods for the determination of stereochemistry and the separation of stereoisomers are well known in the art (e.g., see “Advanced Organic Chemistry”, 4th edition, March, Jerry, John Wiley & Sons, New York, 1992). It is understood that the names and illustration used in this Application to describe compounds of Formula (I) are meant to be encompassed all possible stereoisomers.

“Monosubstituted amino” means a radical —NHR where R is selected from alkyl, hydroxyalkyl, alkoxyalkyl, aminoalkyl, optionally substituted phenyl, optionally substituted phenalkyl, optionally substituted heteroaryl, or optionally substituted heteroaralkyl as defined herein, e.g., methylamino, ethylamino, propylamino, phenylamino, benzylamino, and the like.

“Optional” or “optionally” or “may be” means that the subsequently described event or circumstance may or may not occur, and that the description includes instances where the event or circumstance occurs and instances in which it does not. For example, the phrase “wherein the aromatic ring in R^{a} is optionally substituted with one or two substituents independently selected from alkyl” means that the aromatic ring may or may not be substituted with alkyl in order to fall within the scope of the invention.

The present invention also includes N-oxide derivatives of a compound of Formula (I). N-oxide derivative mean a compound of Formula (I) in which a nitrogen atom is in an oxidized state (i.e., N→O) e.g., pyridine N-oxide, and which possess the desired pharmacological activity.

“Pathology” of a disease means the essential nature, causes and development of the disease as well as the structural and functional changes that result from the disease processes.

“Pharmaceutically acceptable” means that which is useful in preparing a pharmaceutical composition that is generally safe, non toxic and neither biologically nor otherwise undesirable and includes that which is acceptable for veterinary use as well as human pharmaceutical use.

“Pharmaceutically acceptable salts” means salts of compounds of Formula (I) which are pharmaceutically acceptable, as defined above, and which possess the desired pharmacological activity. Such salts include acid addition salts formed with inorganic acids such as hydrochloric acid, hydrobromic acid, sulfuric acid, nitric acid, phosphoric acid, and the like; or with organic acids such as acetic acid, propionic acid, hexanoic acid, heptanoic acid, cyclopentanepropionic acid, glycolic acid, pyruvic acid, lactic acid, malonic acid, succinic acid, malic acid, maleic acid, fumaric acid, tartaric acid, citric acid, benzoic acid, o-(4 hydroxybenzoyl)benzoic acid, cinnamic acid, mandelic acid, methylsulfonic acid, ethanesulfonic acid, 1,2-ethanedisulfonic acid, 2-hydroxy-ethanesulfonic acid, benzenesulfonic acid, p-chlorobenzenesulfonic acid, 2-naphthalenesulfonic acid, p-toluenesulfonic acid, camphorsulfonic acid, 4-methylbicyclo[2.2.2]oct-2-ene-1-carboxylic acid, glucoheptonic acid, 4,4′-methylenebis(3-hydroxy 2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid and the like.

Pharmaceutically acceptable salts also include base addition salts which may be formed when acidic protons present are capable of reacting with inorganic or organic bases. Acceptable inorganic bases include sodium hydroxide, sodium carbonate, potassium hydroxide, aluminum hydroxide and calcium hydroxide. Acceptable organic bases include ethanolamine, diethanolamine, triethanolamine, tromethamine, N-methylglucamine and the like.

The present invention also includes prodrugs of a compound of Formula (I). Prodrug means a compound that is convertible in vivo by metabolic means (e.g. by hydrolysis) to a compound of Formula (I). For example, an ester of a compound of Formula (I) containing a hydroxy group may be convertible by hydrolysis in vivo to the parent molecule. Alternatively an ester of a compound of Formula (I) containing a carboxy group may be convertible by hydrolysis in vivo to the parent molecule. Suitable esters of compounds of Formula (I) containing a hydroxy group, are for example acetates, citrates, lactates, tartrates, malonates, oxalates, salicylates, propionates, succinates, fumarates, maleates, methylene-bis-βb-hydroxynaphthoates, gentisates, isethionates, di-p-toluoyltartrates, methylsulphonates, ethanesulphonates, benzenesulphonates, p-toluenesulphonates, cyclohexylsulphamates and quinates. Suitable esters of compounds of Formula (I) containing a carboxy group, are for example those described by Leinweber, F. J. Drug Metab. Res., 1987, 18, page 379. An especially useful class of esters of compounds of Formula (I) containing a hydroxy group, may be formed from acid moieties selected from those described by Bundgaard et al., J. Med. Chem., 1989, 32, pp 2503-2507, and include substituted (aminomethyl)-benzoates, for example, dialkylamino-methylbenzoates in which the two alkyl groups may be joined together and/or interrupted by an oxygen atom or by an optionally substituted nitrogen atom, e.g. an alkylated nitrogen atom, more especially (morpholino-methyl)benzoates, e.g. 3- or 4-(morpholinomethyl)-benzoates, and (4-alkylpiperazin 1-yl)benzoates, e.g. 3- or 4-(4-alkylpiperazin-1-yl)benzoates.

“Optionally substituted phenyl” means a phenyl ring optionally substituted with one, two, or three substituents independently selected from alkyl, halo, alkoxy, alkylthio, haloalkyl, haloalkoxy, heteroaryl (that is optionally substituted with one or two substituents independently selected from alkyl, halo, hydroxy, alkoxy, carboxy, amino, alkylamino, or dialkylamino), heterocycloalkyl (that is optionally substituted with one or two substituents independently selected from alkyl, halo, hydroxy, alkoxy, carboxy, amino, alkylamino, or dialkylamino), amino, alkylamino, dialkylamino, hydroxy, cyano, nitro, methylenedioxy, hydroxyalkyl, alkoxycarbonyl, or carboxy or optionally substituted with five fluorine atoms.

“Optionally substituted phenyloxy” means a —O—R radical where R is optionally substituted phenyl as defined above e.g., phenyloxy, 4-methoxyphenoxy, and the like.

“Optionally substituted phenylalkyl” or “optionally substituted phenalkyl” means a -(alkylene)-R radical where R is optionally substituted phenyl as defined above e.g., benzyl, phenylethyl, and the like.

“Optionally substituted phenylalkyloxy” means a -(alkylene)-OR radical where R is optionally substituted phenyl as defined above e.g., benzyloxy, fluorophenylethyloxy, and the like.

“Optionally substituted heteroaryl” means a monovalent monocyclic or bicyclic aromatic radical of 5 to 10 ring atoms where one or more, preferably one, two, or three, ring atoms are heteroatoms selected from N, O, or S, the remaining ring atoms being carbon that is optionally substituted with one, two, or three substituents independently selected from alkyl, halo, alkoxy, haloalkyl, haloalkoxy, amino, alkylamino, dialkylamino, hydroxy, cyano, nitro, hydroxyalkyl, alkoxycarbonyl, optionally substituted phenyl, optionally substituted phenoxy, carboxy, or heteroaryl that is optionally substituted with alkyl, halo, hydroxy, alkoxy, carboxy, amino, alkylamino, or dialkylamino, heterocycloalkyl optionally substituted with one or two substituents independently selected from alkyl, halo, hydroxy, alkoxy, amino, alkylamino or dialkylamino, or heterocycloalkylalkyl optionally substituted with one or two substituents independently selected from alkyl, halo, hydroxy, alkoxy, amino, alkylamino or dialkylamino. More specifically the term optionally substituted heteroaryl includes, but is not limited to, pyridyl, pyrrolyl, imidazolyl, thienyl, furanyl, indolyl, quinolyl, pyrazinyl, pyrimidinyl, pyridazinyl, oxazolyl, isoxazolyl, benzoxazolyl, quinolinyl, isoquinolinyl, benzopyranyl, and thiazolyl, and the derivatives thereof (formed when the heteroaryl ring is substituted with a substituent listed above).

“Optionally substituted heteroaryloxy” means a —OR radical where R is optionally substituted heteroaryl as defined above e.g., furanyloxy, pyridinyloxy, and the like.

“Optionally substituted heteroaralkyloxy” means a —OR radical where R is optionally substituted heteroaralkyl ring as defined below.

“Optionally substituted heteroaralkyl” means a -(alkylene)-R radical where R is optionally substituted heteroaryl ring as defined above.

“Optionally substituted heterocycloalkyl” means a saturated or unsaturated monovalent cyclic group of 3 to 8 ring atoms in which one or two ring atoms are heteroatoms selected from N, O, or S(O)ₙ, where n is an integer from 0 to 2, the remaining ring atoms being C. One or two ring carbon atoms can optionally be replaced by a —CO— group. More specifically the term heterocycloalkyl includes, but is not limited to, pyrrolidino, piperidino, morpholino, piperazino, tetrahydropyranyl, and thiomorpholino and the derivatives thereof (formed when the heterocycloalkyl ring is substituted with a substituent listed below); or an N-oxide or a protected derivative thereof. The heterocycloalkyl is optionally fused to aryl and is optionally substituted with one, two, or three substituents independently selected from alkyl, cycloalkyl, halo, alkoxy, haloalkyl, haloalkoxy, amino, alkylamino, dialkylamino, hydroxy, cyano, nitro, optionally substituted phenylalkyl, optionally substituted heteroaralkyl, hydroxyalkyl, alkoxycarbonyl, or carboxy.

“Optionally substituted heterocycloalkyloxy” means a —OR radical where R is optionally substituted heterocycloalkyl ring as defined above.

“Optionally substituted heterocycloalkylalkyl” means a -(alkylene)-R radical where R is optionally substituted heterocycloalkyl ring as defined above.

“Optionally substituted heterocycloalkylalkyloxy” means a —O(alkylene)-R radical where R is optionally substituted heterocycloalkyl ring as defined above.

“Optionally substituted heterocycloalkylalkyloxycarbonyl” means a —C(O)O(alkylene)-R radical where R is optionally substituted heterocycloalkyl ring as defined above.

“Protected derivatives” means derivatives of compounds of Formula (I) in which a reactive site or sites are blocked with protecting groups. Protected derivatives of compounds of Formula (I) are useful in the preparation of compounds of Formula (I) or in themselves may be active cathepsin S inhibitors. A comprehensive list of suitable protecting groups can be found in T. W. Greene, Protective Groups in Organic Synthesis, 3rd edition, John Wiley & Sons, Inc. 1999.

“Sulfonylamino” refers to a —NHSO₂R radical where R is alkyl, hydroxyalkyl, optionally substituted phenyl, optionally substituted heteroaryl, or optionally substituted heterocycloalkyl as defined above e.g., methylsulfonylamino, hydroxyethylsulfonylamino, phenylsulfonylamino, and the like.

“Sulfonylaminoalkyl” refers to a -(alkylene)R radical where R is an sulfonylamino group as defined above e.g., methylsulfonylaminomethyl, hydroxyethylsulfonylaminoethyl, phenylsulfonylaminomethyl, and the like.

“Therapeutically effective amount” means that amount which, when administered to an animal for treating a disease, is sufficient to effect such treatment for the disease.

“Treatment” or “treating” means any administration of a compound of the present invention and includes:
(1) preventing the disease from occurring in an animal which may be predisposed to the disease but does not yet experience or display the pathology or symptomatology of the disease,
(2) inhibiting the disease in an animal that is experiencing or displaying the pathology or symptomatology of the diseased (i.e., arresting further development of the pathology and/or symptomatology), or
(3) ameliorating the disease in an animal that is experiencing or displaying the pathology or symptomatology of the diseased (i.e., reversing the pathology and/or symptomatology).

“Ureido” means a radical —NHCONRR′ where R is hydrogen or alkyl and R′ is hydrogen, alkyl, cycloalkyl, or cycloalkylalkyl.

“Ureidoalkyl” refers to a -(alkylene)R radical where R is a ureido group as defined above.

Representative compounds of Formula (I) where R³ and R⁶ are hydrogen and other groups are as shown in table below are:

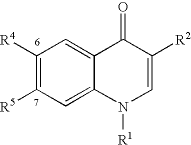

| Cpd.# | R¹ | R² | R⁴ | R⁵ |
|---|---|---|---|---|
| 1 | 4-Cl-benzyl | -C(O)O-(4-Cl-benzyl) | F | piperazin-1-yl |
| 2 | 4-CF₃-benzyl | -C(O)O-(4-Cl-benzyl) | F | morpholin-4-yl |
| 3 | 4-Cl-benzyl | -C(O)O-(4-Cl-benzyl) | F | morpholin-4-yl |
| 4 | 4-Cl-benzyl | -C(O)O-(4-CF₃-benzyl) | F | morpholin-4-yl |
| 5 | 2-F-4-CF₃-benzyl | -C(O)-(4-CH₃-phenyl) | OCH₃ | OCH₃ |
| 6 | 4-SO₂CH₃-benzyl | -C(O)-(4-CH₃-phenyl) | OCH₃ | OCH₃ |
| 7 | 4-C(CH₃)₃-benzyl | -C(O)-(4-CH₃-phenyl) | OCH₃ | OCH₃ |
| 8 | 4-SO₂CH₃-benzyl | -C(O)-(4-CH₃-phenyl) | OH | OH |
| 9 | 4-Cl-benzyl | -C(O)-(4-CH₃-phenyl) | OCH₃ | OCH₃ |
| 10 | 3,4-diCl-benzyl | -C(O)-(4-CH₃-phenyl) | OCH₃ | OCH₃ |
| 11 | 3-OCF₃-benzyl | -C(O)-(4-CH₃-phenyl) | OCH₃ | OCH₃ |
| 12 | 4-SO₂CH₃-benzyl | -C(O)-(4-CH₃-phenyl) | Cl | H |
| 13 | 4-OCHF₂-benzyl | -C(O)-(4-CH₃-phenyl) | OCH₃ | OCH₃ |
| 14 | 4-NO₂-benzyl | -C(O)-(4-CH₃-phenyl) | OCH₃ | OCH₃ |
| 15 | benzo[1.2.5]oxadiazol-4- | -C(O)-(4-CH₃-phenyl) | OCH₃ | OCH₃ |
| | ylmethyl | | | |
| 16 | 3-phenpropyl | -C(O)-(4-CH₃-phenyl) | OCH₃ | OCH₃ |
| 17 | 4-SO₂CH₃-benzyl | -C(O)-(4-CH₃-phenyl) | H | OCH₃ |
| 18 | 4-CH(CH₃)₂-benzyl | -C(O)-(5-CH₃-pyridin-2-yl) | OCH₃ | OCH₃ |
| 19 | 4-SO₂CH₃-benzyl | -C(O)-(4-CH₃-phenyl) | CONH₂ | H |
| 20 | 4-CH₃-benzyl | -C(O)-(4-OCH₃-phenyl) | OCH₃ | OCH₃ |
| 21 | 4-CH₃-benzyl | -C(O)-(3,4-diCH₃-phenyl) | OCH₃ | OCH₃ |
| 22 | 2-Cl-pyridin-5-ylmethyl | -C(O)-(4-CH₃-phenyl) | OCH₃ | OCH₃ |
| 23 | 4-CF₃-benzyl | -C(O)-(4-CH₃-piperidin-1-yl) | OCH₃ | OCH₃ |
| 24 | 4-NHCOCH₃-benzyl | -C(O)-(4-CH₃-phenyl) | OCH₃ | OCH₃ |
| 25 | 4-CN-benzyl | -C(O)-(4-CH₃-phenyl) | OCH₃ | OCH₃ |
| 26 | 4-indol-2-ylmethyl (?) | -C(O)-(4-CH₃-phenyl) | OCH₃ | OCH₃ |
| | wrong name | | | |
| 27 | cyclohexylmethyl | -C(O)-(4-CH₃-phenyl) | OCH₃ | OCH₃ |
| 28 | 3,4-diCl-benzyl | -CH(OH)-(4-CH₃-phenyl) | OCH₃ | OCH₃ |
| 29 | 4-CH(CH₃)₂-benzyl | -C(O)-(piperidin-1-yl) | OCH₃ | OCH₃ |
| 30 | 4-SO₂CH₃-benzyl | -CHOH-(4-CH₃-phenyl) | OCH₃ | OCH₃ |
| 31 | 3-CN-benzyl | -C(O)-(4-CH₃-phenyl) | OCH₃ | OCH₃ |
| 32 | 4-SO₂CH₃-benzyl | -C(O)-(4-CH₃-phenyl) | H | CN |
| 33 | 4-Cl-benzyl | -C(O)NH-(4-Cl-phenyl) | F | morpholin-4-yl |
| 34 | 3-COOCH₃-benzyl | -C(O)-(4-CH₃-phenyl) | OCH₃ | OCH₃ |
| 35 | 4-CH(CH₃)₂-benzyl | -C(O)-(phenyl) | OCH₃ | OCH₃ |
| 36 | 4-CF₃-benzyl | -C(O)-(piperidin-1-yl) | OCH₃ | OCH₃ |
| 37 | 4-SO₂CH₃-benzyl | -C(O)-[2-N(CH₃)₂pyridin-5-yl] | OCH₃ | OCH₃ |
| 38 | 4-SO₂CH₃-benzyl | -C(O)-(4-F-phenyl) | OCH₃ | OCH₃ |
| 39 | 4-C(CH₃)₃-benzyl | -C(O)-(pyridin-4-yl) | OCH₃ | OCH₃ |
| 40 | 4-C(CH₃)₃-benzyl | -C(O)-(2-Clpyridin-5-yl) | OCH₃ | OCH₃ |
| 41 | 2-(NHC₂H₅)-thiazol- | -C(O)-(4-CH₃-phenyl) | OCH₃ | OCH₃ |
| | 4ylmethyl | | | |
| 42 | 4-COOCH₃-benzyl | -C(O)-(4-CH₃-phenyl) | OCH₃ | OCH₃ |
| 43 | 4-SO₂CH₃-benzyl | -C(O)-[2-Cl pyridin-5-yl] | OCH₃ | OCH₃ |
| 44 | 1-CH₃benzotriazol-5- | -C(O)-(4-CH₃-phenyl) | OCH₃ | OCH₃ |
| | ylmethyl | | | |
| 45 | 4-SO₂CH₃-benzyl | -C(O)-[3-Brpyridin-5-yl] | OCH₃ | OCH₃ |
| 46 | 3-CF₃-benzyl | -C(O)-(4-CH₃-phenyl) | OCH₃ | OCH₃ |
| 47 | 2-phenethyl | -C(O)-(4-CH₃-phenyl) | OCH₃ | OCH₃ |
| 48 | cyclopropylmethyl | -C(O)-(4-CH₃-phenyl) | OCH₃ | OCH₃ |
| 49 | 4-NH₂-benzyl | -C(O)-(4-CH₃-phenyl) | OCH₃ | OCH₃ |
| 50 | 4-CF₃-benzyl | -C(O)-(4-Bocpiperizin-1-yl) | OCH₃ | OCH₃ |
| 51 | 4-Cl-benzyl | -C(O)-(1,2,3,4-tetrahydro- | F | morpholin-4-yl |
| | | isoquinolin-1-yl) | | |
| 52 | 4-CN-benzyl | -C(O)O-(4-Cl-benzyl) | OMe | OMe |
| 53 | 4-CF3-benzyl | -C(O)O-(4-CH3-benzyl) | OMe | OMe |
| 54 | 2-F-4-CF3-benzyl | -C(O)O-(4-CH3-benzyl) | OMe | OMe |
| 55 | 2-F-4-CF3-benzyl | -C(O)O-(2-CH3-benzyl) | OMe | OMe |
| 56 | 2-F-4-CF3-benzyl | -C(O)O-(3-CH3-benzyl) | OMe | OMe |
| 57 | 2-F-4-CF3-benzyl | -C(O)O-(3-Cl-benzyl) | OMe | OMe |
| 58 | 2-F-4-CF3-benzyl | -C(O)O-(2-OCH3-benzyl) | OMe | OMe |
| 59 | 2-F-4-CF3-benzyl | -C(O)O-(2-Cl-benzyl) | OMe | OMe |
| 60 | 2-F-4-CF3-benzyl | -C(O)O-(3-OCH3-benzyl) | OMe | OMe |
| 61 | 2-F-4-CF3-benzyl | -C(O)O-(2,5-diCl-benzyl) | OMe | OMe |
| 62 | 4-CH3SO2-benzyl | benzyl | F | N-Me- |
| | | | | morpholin-4-yl |
| 63 | 4-CH3SO2-benzyl | 4-Cl-benzyl | F | N-Me- |
| | | | | morpholin-4-yl |
| 64 | 4-CH3SO2-benzyl | 4-CH3-benzyl | F | N-Me- |
| | | | | morpholin-4-yl |

### PREFERRED EMBODIMENTS

Certain compounds of Formula (I) within the broadest scope set forth in the Summary of the Invention are preferred. For example:

A. A preferred group of compounds of Formula (I) is that wherein:

R¹ is -(alkylene)-R⁷ where R⁷ is aryl optionally substituted with one, two, or three R^{a}. Preferably R¹ is benzyl or 3-phenylpropyl optionally substituted with one, two, or three R^{a} independently selected from halo, alkyl, haloalkyl, haloalkoxy, alkylsulfonyl, cyano, or alkoxycarbonyl, more preferably benzyl optionally substituted with halo, even more preferably benzyl or 4-chlorobenzyl; and

R⁶ is hydrogen, alkyl, or halo, preferably hydrogen.

B. Another preferred group of compounds of Formula (I) is that wherein:

R¹ is -(alkylene)-R⁷ where R⁷ is heteroaryl optionally substituted with one, two, or three R^{a}; and

R⁶ is hydrogen, alkyl, or halo, preferably hydrogen.

C. Yet another preferred group of compounds of Formula (I) is that wherein:

R¹ is -(alkylene)-R⁷ where R⁷ is heterocycloalkyl optionally substituted with one, two, or three R^{a}; and

R⁶ is hydrogen, alkyl, or halo, preferably hydrogen.

D. Yet another preferred group of compounds of Formula (I) is that wherein R⁴ is halo and R⁵ is heterocycloalkyl, preferably pyrroldinyl, piperidinyl, piperazinyl, or morpholinyl optionally substituted with one, two, or three R^{d}.

E. Yet another preferred group of compounds of Formula (I) is that wherein:

R¹ is -(alkylene)-R⁷ where R⁷ is cycloalkyl optionally substituted with one or two, preferably cyclohexylmethyl; and

R⁶ is hydrogen, alkyl, or halo, preferably hydrogen.

1. Within the above preferred groups A, B and C and more preferred groups contained therein, a more preferred group of compounds is that wherein:

R² is —COR⁸ where R⁸ is aryl, aralkyl, heteroaryl, or heteroaralkyl optionally substituted with one, two, or three R^{b}. Preferably, R⁸ is phenyl optionally substituted with one, two, or three R^{b} independently selected from halo, alkyl, haloalkyl, haloalkoxy, aminosulfonyl, or alkylsulfonyl.

2. Within the above preferred groups A, B and C and more preferred groups contained therein, another more preferred group of compounds is that wherein:

R² is —COOR⁹ where R⁹ is alkyl, aryl, aralkyl, heteroaryl, or heteroaralkyl optionally substituted with one, two, or three R^{b}. Preferably, R⁹ is aralkyl optionally substituted with one, two, or three R^{b}, more preferably —C(O)OCH₂phenyl or —C(O)C(CH3)₂phenyl optionally substituted with one, two, or three R^{b}, preferably halo, even more preferably R⁹ is —C(O)OCH₂ (4-Clphenyl).

3. Within the above preferred groups A, B and C and more preferred groups contained therein, another more preferred group of compounds is that wherein R² is —CONR^{10 R}¹¹ where R¹⁰ hydrogen, alkyl, aryl, aralkyl, heteroaryl, or heteroaralkyl and R¹¹ is alkyl, aryl, aralkyl, heteroaryl, or heteroaralkyl optionally substituted with one, two, or three R^{b}.

4. Within the above preferred groups A, B and C and more preferred groups contained therein, another more preferred group of compounds is that wherein R² is —CONR¹⁰R¹¹ where R¹⁰ and R¹¹ together with the nitrogen atom to which they are attached form heterocycloamino, preferably piperidinyl, piperazinyl, or pyrrolidinyl optionally substituted with one, two, or three R^{b}.

5. Within the above preferred groups A, B and C and more preferred groups contained therein, another more preferred group of compounds is that wherein R² is —NR¹²R¹³ where R¹² is hydrogen, alkyl, hydroxyalkyl, alkoxyalkyl, aryl, aralkyl, heteroaryl, heteroaralkyl, or heterocycloalkylalkyl and R¹³ is hydrogen, alkyl, aryl, aralkyl, heteroaryl, heteroaralkyl, or heterocycloalkylalkyl optionally substituted with one, two, or three R^{b}.

6. Within the above preferred groups A, B and C and more preferred groups contained therein, another more preferred group of compounds is that wherein R² is —SO₂NR¹⁶R¹⁷ where R¹⁶ hydrogen, alkyl, aryl, aralkyl, heteroaryl, or heteroaralkyl and R¹⁷ is alkyl, aryl, aralkyl, heteroaryl, or heteroaralkyl optionally substituted with one, two, or three R^{b}.

7. Within the above preferred groups A, B and C and more preferred groups contained therein, another more preferred group of compounds is that wherein R² is —NHSO₂R¹⁸ where R¹⁸ is aryl, aralkyl, heteroaryl, heteroaralkyl, or heterocycloalkyl optionally substituted with one, two, or three R^{b}.

8. Within the above preferred groups A, B and C and more preferred groups contained therein, another more preferred group of compounds is that wherein R² is —NHCOOR¹⁹ where R¹⁹ is aryl, aralkyl, heteroaryl, heteroaralkyl, or heterocycloalkyl optionally substituted with one, two, or three R^{b}.

9. Within the above preferred groups A, B and C and more preferred groups contained therein, another more preferred group of compounds is that wherein R² is —NHCONHR²⁰R²¹ where R²⁰ is hydrogen or alkyl and R²¹ is aryl, aralkyl, heteroaryl, heteroaralkyl, or heterocycloalkyl or R²⁰ and R²¹ together with the nitrogen atom to which they are attached form heterocycloamino optionally substituted with one, two, or three R^{b}.

10. Within the above preferred groups A, B and C and more preferred groups contained therein, another more preferred group of compounds is that wherein R² is —SR²² where R²² is aryl, aralkyl, heteroaryl, or heteroaralkyl optionally substituted with one, two, or three R^{b}.

11. Within the above preferred groups A, B and C and more preferred groups contained therein, another more preferred group of compounds is that wherein R² is —SO₂R²³ where R²³ is aryl, aralkyl, heteroaryl, or heteroaralkyl), or —SO₃R²⁴ where R²⁴ is aryl, aralkyl, heteroaryl, or heteroaralkyl optionally substituted with one, two, or three R^{b}.

(i) Within the above preferred groups A, A(1-6), B, B(1-6), C, and C(1-6) and more preferred groups contained therein an even more preferred group of compounds is that wherein R³ is hydrogen or alkoxy and R⁴ and R⁵ are as defined in the Summary of the Invention. Preferably, R⁴ and R⁵ are located at C-6 and C-7 positions of the quinolone ring. Preferably, R³ is located at the C-8 of the quinoline ring, R⁴ is located at the C-6 position of the quinoline ring and is selected from halo, aminocarbonyl, aminocarbonylalkyloxy, hydroxyl, or alkoxy, preferably fluoro, methoxy or —CONH₂ and R⁵ is located at C-7 positions of the quinolone ring and is heterocycloalkyl, preferably piperizin-1-yl, piperidin-2-yl, morpholin-4-yl or homopiperazine wherein the heterocycloalkyl ring is optionally substituted with one, two, or three R^{d}.

General Synthetic Scheme

Compounds of this invention can be made by the methods depicted in the reaction schemes shown below.

The starting materials and reagents used in preparing these compounds are either available from commercial suppliers such as Aldrich Chemical Co., (Milwaukee, Wis.), Bachem (Torrance, Calif.), or Sigma (St. Louis, Mo.) or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), March's Advanced Organic Chemistry, (John Wiley and Sons, 4th Edition) and Larock's Comprehensive Organic Transformations (VCH Publishers Inc., 1989). These schemes are merely illustrative of some methods by which the compounds of this invention can be synthesized, and various modifications to these schemes can be made and will be suggested to one skilled in the art having referred to this disclosure.

The starting materials and the intermediates of the reaction may be isolated and purified if desired using conventional techniques, including but not limited to filtration, distillation, crystallization, chromatography and the like. Such materials may be characterized using conventional means, including physical constants and spectral data. Unless specified to the contrary, the reactions described herein take place at atmospheric pressure over a temperature range from about −78° C. to about 150° C., more preferably from about 0° C. to about 125° C. and most preferably at about room (or ambient) temperature, e.g., about 20° C.

In the reactions described hereinafter it may be necessary to protect reactive functional groups, for example hydroxy, amino, imino, thio or carboxy groups, where these are desired in the final product, to avoid their unwanted participation in the reactions. Conventional protecting groups may be used in accordance with standard practice, for examples see T. W. Greene and P. G. M. Wuts in “Protective Groups in Organic Chemistry” John Wiley and Sons, 1999.

Compounds of Formula (I) where R² is —COOR⁹ or —CONR¹⁰R¹¹, R⁶ is hydrogen, and other groups are as defined in the Summary of the Invention can be prepared by proceeding as in the following Reaction Scheme 1 below.

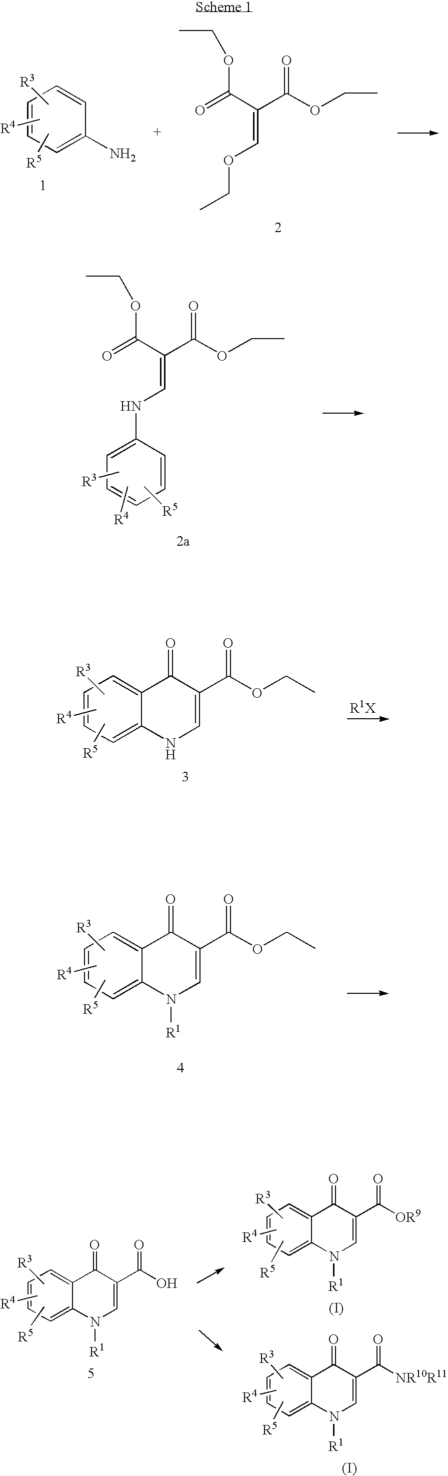

Reaction of an aniline of formula 1 where R³, R⁴ and R⁵ are as defined in Summary of the Invention with 2-ethoxymethylenemalonic acid diethyl ester in refluxing alcoholic solution such as isopropanol provides a compound of formula 2a which upon heating in a high boiling ethereal solvent such diphenylether provides a compound of formula 3. Compound of formula 1 and 3 such as aniline, difluoroaniline, dimethylaniline, dimethoxyaniline, 2-ethoxymethylenemalonic acid diethyl ester are commercially available.

Reaction of a compound of formula 3 with an alkylating agent of formula R¹X where R¹ is as defined in the Summary of the Invention and X is a leaving group such as halo, tosylate, mesylate and the like provides a compound of formula 4. The reaction is typically carried out in the presence of a base such as potassium carbonate, cesium carbonate, sodium hydride, and the like and in a suitable organic solvent such as dimethylformamide, tetrahydrofuran, and the like.

Hydrolysis of the ester group in 4 under basic hydrolysis reaction conditions provides a compound of formula 5. Compound 5 is then converted to a compound of Formula (I) where R² is —COOR⁹ where R⁹ is other than ethyl by reacting 5 wth an alcohol of formula R⁹OH where R⁹ is as defined in the Summary of the Invention in the presence of a base such as dimethylaminopyridine and an activating agent such as DCC and in an organic solvent such as dimethylformamde, and the like. Compound of formula 5 can be converted to a compound of Formula (I) where R² is —CONR¹⁰R¹¹ by coupling it with an amine of formula NHR¹⁰R¹¹.

The reaction is typically carried out in the presence of a suitable coupling agent e.g., benzotriazole-1-yloxytrispyrrolidinophosphonium hexafluorophosphate (PyBOP®), O-benzotriazol-1-yl-N,N,N′,N′-tetramethyl-uronium hexafluorophosphate (HBTU), O-(7-azabenzotriazol-1-yl) 1,1,3,3-tetramethyl-uronium hexafluorophosphate (HATU), 1-(3-dimethylaminopropyl)-3-ethylcarbodiimide hydrochloride (EDC), or 1,3-dicyclohexyl-carbodiimide (DCC), optionally in the presence of 1-hydroxy-benzotriazole (HOBT), and a base such as N,N-diisopropylethylamine, triethylamine, N-methylmorpholine, and the like. Suitable reaction solvents are inert organic solvents such as halogenated organic solvents (e.g., methylene chloride, chloroform, and the like), acetonitrile, N,N-dimethylformamide, ethereal solvents such as tetrahydrofuran, dioxane, and the like or mixtures thereof. Compounds of formula R⁹OH and NHR¹⁰R¹¹ are commercially available or they can be prepared by methods well known in the art.

Compounds of Formula (I) where R² is —COR⁸ or —C(OH)R⁸R¹⁴, R⁶ is hydrogen, and other groups are as defined in the Summary of the Invention can be prepared by proceeding as in the following Reaction Scheme 2 below.

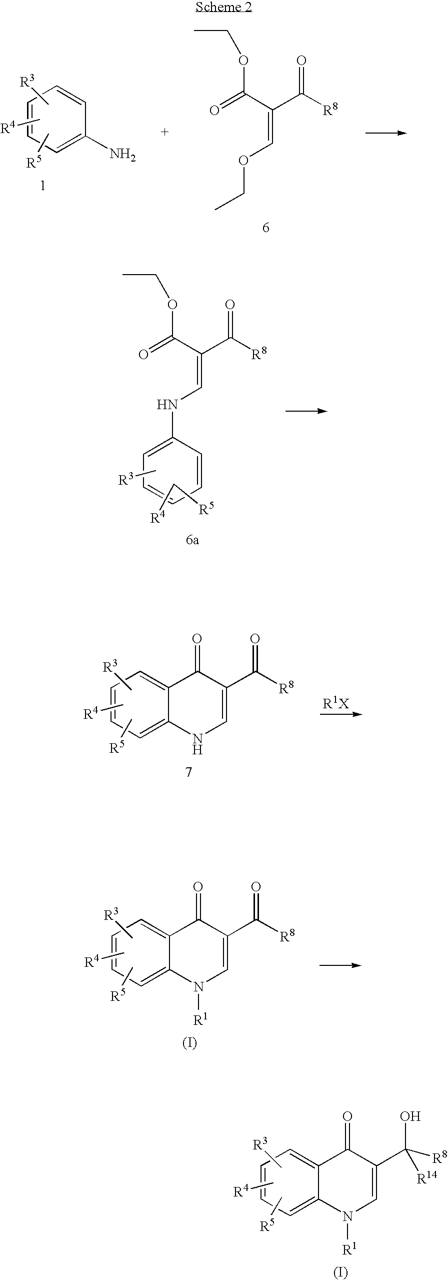

Reaction of an aniline of formula 1 with 3-ethoxyacrylic acid ethyl ester of formula 6 as described above provides a compound of formula 6a which is then converted to a 3-ketoquinolone compound of formula 7 which is then converted to a compound of Formula (I) as described above. Compound of formula 6 can be prepared by treating a beta-ketoester of formula R⁸COCH₂COEt with triethylorthoformate and acetic anhydride. Beta ketoester of formula R⁸CO—CH₂COEt are either commercially available or they can be prepared by methods described in Tetrahedron Letters, 35, 9323, 1994. A compound of Formula (I) where R² is ketone group can be converted to a corresponding compound of Formula (I) where R² is —CH(OH)R⁸ by reducing the ketone group with a suitable reducing agent such as sodium borohydride, and the like. Compounds of Formula (I) where R² is —C(OH)R⁸R¹⁴ where R¹⁴ is other than hydrogen can be prepared by treating a compound of Formula (I) where R² is ketone group with an organometallic reagent such as R¹⁴MgX or R¹⁴Li in a suitable organic solvent such as tetrahydrofuran and the like. R¹⁴MgX or R¹⁴Li are either commericially available or can be prepared by methods well known in the art.

Compounds of Formula (I) where R² is —SO₂NR¹⁶R¹⁷, R⁶ is hydrogen, and other groups are as defined in the Summary of the Invention can be prepared by proceeding as in the following Reaction Scheme 3 below.

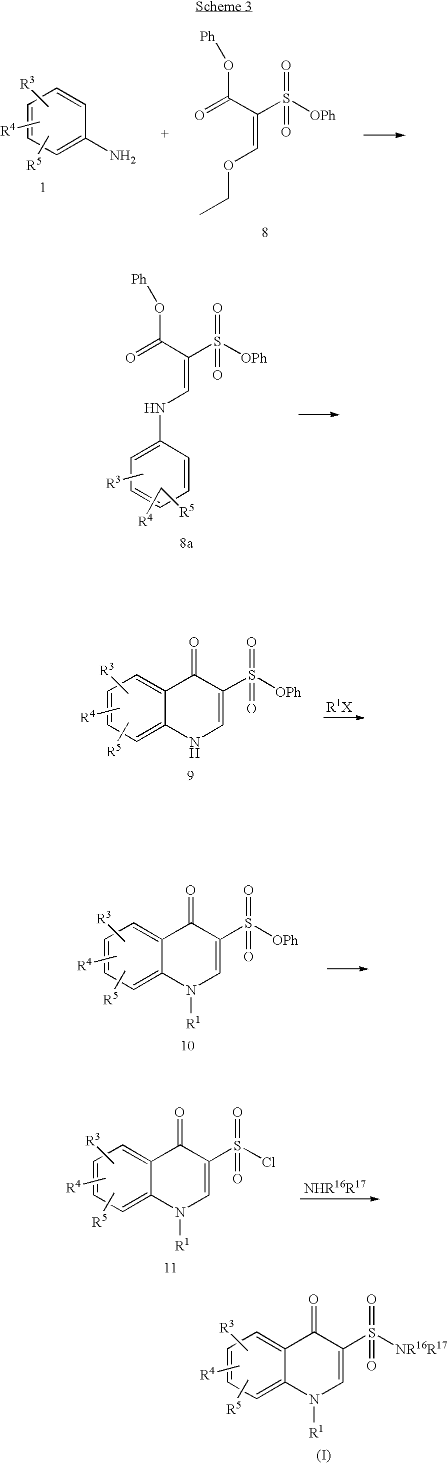

Treatment of an aniline of formula 1 with a compound of formula 8 under the reaction conditions described in Scheme 1 above provides a compound of formula 8a which is then converted to a compound of formula 10 as described above. Compound of formula 8 can be prepared by heating chlorosulfonylacetyl chloride in phenol at 110° C.

Alkaline hydrolysis of the compound 10 with 10% aq NaOH followed by conversion of resultant sulfonic acid 10 A to sulfonyl chloride 11 employing reagents such as POCl₃ or thionyl chloride in solvents such as dichloromethane. The sulfonyl chloride 11 upon treatment with an amine of formula NHR¹⁶R¹⁷ in solvents such as dichloromethane furnishes compounds of formula (I).

Compound of formula 11 can be reacted with a hydroxyl compound of formula R²⁴OH to provide a compound of Formula (I) where R² is —SO₃R²⁴.

A compound of Formula (I) can be converted to other compounds of Formula (I). For example:

A compound of Formula (I) containing a hydroxy group may be prepared by de-alkylation/benzylation of an alkoxy/benzyloxy substituent; those containing an acid group, by hydrolysis of an ester group; and those containing a cyano, by displacement of a bromine atom on the corresponding compounds of Formula (I). A compound of Formula (I) containing a cyano group can be converted to a corresponding carboxy containing compound by hydrolysis of the cyano group. The carboxy group, in turn, can be converted to an ester group. A compound of Formula (I) where R⁵ is heterocycloalkyl can be prepared from a corresponding compound of Formula (I) where R⁵ is halo, preferably fluoro, by heating it with the desired heterocycloalkyl in a suitable organic solvent such as acetonitrile.

A compound of Formula (I) can be prepared as a pharmaceutically acceptable acid addition salt by reacting the free base form of the compound with a pharmaceutically acceptable inorganic or organic acid. Alternatively, a pharmaceutically acceptable base addition salt of a compound of Formula (I) can be prepared by reacting the free acid form of the compound with a pharmaceutically acceptable inorganic or organic base. Inorganic and organic acids and bases suitable for the preparation of the pharmaceutically acceptable salts of compounds of Formula (I) are set forth in the definitions section of this Application. Alternatively, the salt forms of the compounds of Formula (I) can be prepared using salts of the starting materials or intermediates.

The free acid or free base forms of the compounds of Formula (I) can be prepared from the corresponding base addition salt or acid addition salt form. For example, a compound of Formula (I) in an acid addition salt form can be converted to the corresponding free base by treating with a suitable base (e.g., ammonium hydroxide solution, sodium hydroxide, and the like). A compound of Formula (I) in a base addition salt form can be converted to the corresponding free acid by treating with a suitable acid (e.g., hydrochloric acid, etc).

The N-oxides of compounds of Formula (I) can be prepared by methods known to those of ordinary skill in the art. For example, N-oxides can be prepared by treating an unoxidized form of the compound of Formula (I) with an oxidizing agent (e.g., trifluoroperacetic acid, permaleic acid, perbenzoic acid, peracetic acid, meta-chloroperoxybenzoic acid, or the like) in a suitable inert organic solvent (e.g., a halogenated hydrocarbon such as dichloromethane) at approximately 0° C. Alternatively, the N-oxides of the compounds of Formula (I) can be prepared from the N-oxide of an appropriate starting material.

Compounds of Formula (I) in unoxidized form can be prepared from N-oxides of compounds of Formula (I) by treating with a reducing agent (e.g., sulfur, sulfur dioxide, triphenyl phosphine, lithium borohydride, sodium borohydride, phosphorus trichloride, tribromide, or the like) in a suitable inert organic solvent (e.g., acetonitrile, ethanol, aqueous dioxane, or the like) at 0 to 80° C.

Prodrug derivatives of the compounds of Formula (I) can be prepared by methods known to those of ordinary skill in the art (e.g., for further details see Saulnier et al. (1994), Bioorganic and Medicinal Chemistry Letters, Vol. 4, p. 1985). For example, appropriate prodrugs can be prepared by reacting a non derivatized compound of Formula (I) with a suitable carbamylating agent (e.g., 1,1-acyloxyalkylcarbonochloridate, para-nitrophenyl carbonate, or the like).

Protected derivatives of the compounds of Formula (I) can be made by means known to those of ordinary skill in the art. A detailed description of the techniques applicable to the creation of protecting groups and their removal can be found in T. W. Greene, Protecting Groups in Organic Synthesis, 3^{rd} edition, John Wiley & Sons, Inc. 1999.

Compounds of the present invention may be conveniently prepared or formed during the process of the invention, as solvates (e.g. hydrates). Hydrates of compounds of the present invention may be conveniently prepared by recrystallisation from an aqueous/organic solvent mixture, using organic solvents such as dioxin, tetrahydrofuran or methanol.

Compounds of Formula (I) can be prepared as their individual stereoisomers by reacting a racemic mixture of the compound with an optically active resolving agent to form a pair of diastereoisomeric compounds, separating the diastereomers and recovering the optically pure enantiomer. While resolution of enantiomers can be carried out using covalent diasteromeric derivatives of compounds of Formula (I), dissociable complexes are preferred (e.g., crystalline diastereoisomeric salts). Diastereomers have distinct physical properties (e.g., melting points, boiling points, solubilities, reactivity, etc.) and can be readily separated by taking advantage of these dissimilarities. The diastereomers can be separated by chromatography or, preferably, by separation/resolution techniques based upon differences in solubility. The optically pure enantiomer is then recovered, along with the resolving agent, by any practical means that would not result in racemization. A more detailed description of the techniques applicable to the resolution of stereoisomers of compounds from their racemic mixture can be found in Jean Jacques Andre Collet, Samuel H. Wilen, Enantiomers, Racemates and Resolutions, John Wiley & Sons, Inc. (1981).

Pharmacology and Utility

The compounds of the present invention inhibit replication of HCV and are therefore useful in treating hepatitis C infections.

The inhibitory activities of the compounds of Formula (I) can be determined by methods known to those of ordinary skill in the art. Suitable in vitro assays for measuring protease activity and the inhibition thereof by test compounds are known. Details of assays for measuring inhibition of HCV replication are set forth in Biological Examples 1, infra.

Administration and Pharmaceutical Compositions

In general, compounds of Formula (I) will be administered in therapeutically effective amounts via any of the usual and acceptable modes known in the art, either singly or in combination with one or more therapeutic agents. A therapeutically effective amount may vary widely depending on the severity of the disease, the age and relative health of the subject, the potency of the compound used and other factors. For example, therapeutically effective amounts of a compound of Formula (I) may range from about 10 micrograms per kilogram body weight (μg/kg) per day to about 100 milligram per kilogram body weight (mg/kg) per day, typically from about 100 μg/kg/day to about 10 mg/kg/day. Therefore, a therapeutically effective amount for an 80 kg human patient may range from about 1 mg/day to about 8 g/day, typically from about 1 mg/day to about 800 mg/day. In general, one of ordinary skill in the art, acting in reliance upon personal knowledge and the disclosure of this Application, will be able to ascertain a therapeutically effective amount of a compound of Formula (I) for treating a given disease.

The compounds of Formula (I) can be administered as pharmaceutical compositions by one of the following routes: oral, systemic (e.g., transdermal, intranasal or by suppository) or parenteral (e.g., intramuscular, intravenous or subcutaneous). Compositions can take the form of tablets, pills, capsules, semisolids, powders, sustained release formulations, solutions, suspensions, elixirs, aerosols, or any other appropriate composition and are comprised of, in general, a compound of Formula (I) in combination with at least one pharmaceutically acceptable excipient. Acceptable excipients are non toxic, aid administration, and do not adversely affect the therapeutic benefit of the active ingredient. Such excipient may be any solid, liquid, semisolid or, in the case of an aerosol composition, gaseous excipient that is generally available to one of skill in the art.

Solid pharmaceutical excipients include starch, cellulose, talc, glucose, lactose, sucrose, gelatin, malt, rice, flour, chalk, silica gel, magnesium stearate, sodium stearate, glycerol monostearate, sodium chloride, dried skim milk, and the like. Liquid and semisolid excipients may be selected from water, ethanol, glycerol, propylene glycol and various oils, including those of petroleum, animal, vegetable or synthetic origin (e.g., peanut oil, soybean oil, mineral oil, sesame oil, and the like). Preferred liquid carriers, particularly for injectable solutions, include water, saline, aqueous dextrose and glycols.

The amount of a compound of Formula (I) in the composition may vary widely depending upon the type of formulation, size of a unit dosage, kind of excipients and other factors known to those of skill in the art of pharmaceutical sciences. In general, a composition of a compound of Formula (I) for treating a given disease will comprise from 0.01% w to 90% w, preferably 5% w to 50% w, of active ingredient with the remainder being the excipient or excipients. Preferably the pharmaceutical composition is administered in a single unit dosage form for continuous treatment or in a single unit dosage form ad libitum when relief of symptoms is specifically required. Representative pharmaceutical formulations containing a compound of Formula (I) are described below.

In some embodiments, the compounds of Formula (I) can be administered to a patient in need of treatement with a second antiviral agent. Examples of suitable antiviral agents are interferons, such as Intron A, Roferon A and pegylated interferons such as PEG-intron, Pegasys; Ribavirin, Viramidine, Levovirin; HCV polymerase inhibitors such as Valopicitabine, R 1626 (Roche), HCV-796 (Viropharma/Wyeth); and toll receptor agonists such as ANA-975 (Anadys). The compounds of Formula (I) can be administered in a combination with the above agents or separately. Additionally, the compounds of Formula (I) can be administered either prior to, or following, the administration of a second antiviral agent, according to a physician prescribed regimen.

### EXAMPLES

The present invention is further exemplified, but not limited by, the following examples that illustrate the preparation of compounds of Formula (I) according to the invention.

### Example 1

### Synthesis of 1-(4-chlorobenzyl)-6-fluoro-4-oxo-7-piperazin-1-yl-1,4-dihydro-quinoline-3-carboxylic acid 4-chlorobenzyl ester

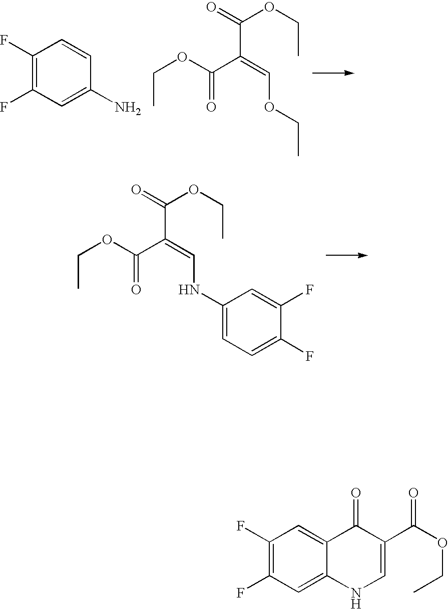 Step 1

To a magnetically stirred solution of 2-ethoxymethylenemalonic acid diethyl ester (5 g, 23.1 mmol) in isopropanol (75 mL) was added 3,4-difluoroaniline (2.98 g, 15 mmol) and refluxed for 4-6 hr. The reaction was monitored by HPLC and mass spec. Once the reaction was complete, solvent was evaporated and the residue was purified with ethylacetate/hexane on a CombiFlash chromatographic system to give 2-[(3,4-difluorophenylaminomethylene]malonic acid diethyl ester.

Step 2

To the refluxing diphenyl ether (60 mL) was added slowly intermediate A (5 g, 16.7 mmol) (either as solid or solution in diphenyl ether) and the reaction mixture was allowed to reflux for 30-60 min. Once the reaction was complete, the reaction mixture to come to room temperature to give 6,7-difluoro-4-oxo-1,4-dihydroquinoline-3-carboxylic acid ethyl ester as a pale yellow solid. 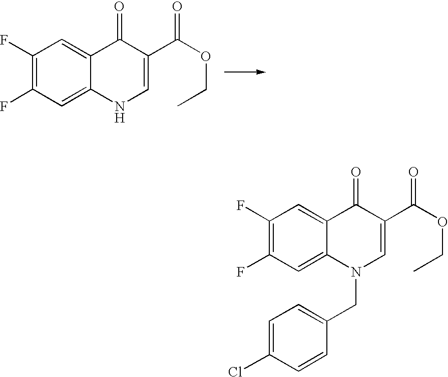
Step 3

To a stirred solution of 6,7-difluoro-4-oxo-1,4-dihydroquinoline-3-carboxylic acid ethyl ester (500 mg, 1.97 mmol) in DMF was added potassium carbonate (545 mg, 3.95 mmols) and 4-chlorobenzylbromide (604 mg, 2.95 mmols) and the reaction mixture was heated at 60° C. for 2 hrs. Once the reaction was complete, the solvent was evaporated on the rotovap and purified by Combiflash using ethyl acetate and hexane as eluent to give 1-(4-chlorobenzyl)-6,7-difluoro-4-oxo-1,4-dihydroquinoline-3-carboxylic acid ethyl ester. 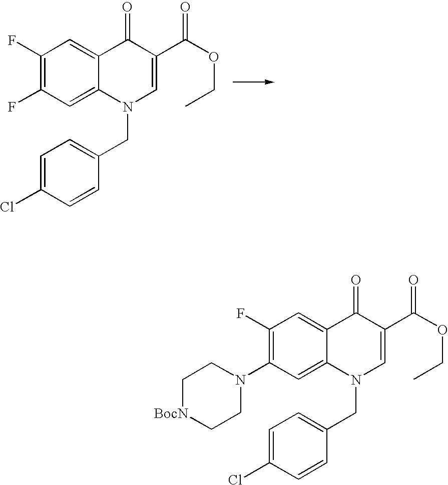
Step 4:

To magnetically stirred solution of 1-(4-chlorobenzyl)-6,7-difluoro-4-oxo-1,4-dihydro-quinoline-3-carboxylic acid ethyl ester (500 mg, 1.32 mmols) in acetonitrile was added N-Bocpiperazine (1.3 gm, 6.63 mmols), and the reaction mixture was heated to reflux overnight. Solvent was evaporated and product was purified by silica gel chromatography with ethylacetate and hexane as eluents to give 7-(4-tert-butoxycarbonylpiperazin-1-yl)-1-(4-chlorobenzyl)-6-fluoro-4-oxo-1,4-dihydroquinoline-3-carboxylic acid ethyl ester. m/z: 544 (M+1) 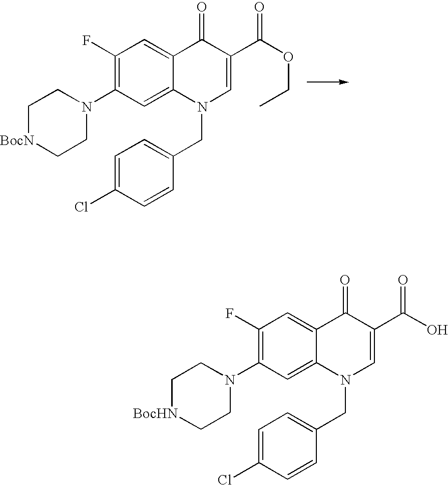
Step 5:

To a magnetically stirred solution of 7-(4-tert-butoxycarbonylpiperazin-1-yl)-1-(4-chlorobenzyl)-6-fluoro-4-oxo-1,4-dihydroquinoline-3-carboxylic acid ethyl ester (500 mg, 0.91 mmols) in MeOH was added 50% aq. NaOH and MeOH (5 mL each) and the reaction mixture was heated to reflux for 3 hrs. The reaction mixture was allowed to cool to room temperature to give the sodium salt of 7-(4-tert-butoxycarbonylpiperazin-1-yl)-1-(4-chlorobenzyl)-6-fluoro-4-oxo-1,4-dihydroquinoline-3-carboxylic acid as a white solid having purity >95%. The above product was stirred in 4 M HCl (10 mL) for 10 min and filtered to give 7-(4-tert-butoxycarbonylpiperazin-1-yl)-1-(4-chlorobenzyl)-6-fluoro-4-oxo-1,4-dihydroquinoline-3-carboxylic acid which was used in the next step without further purification. 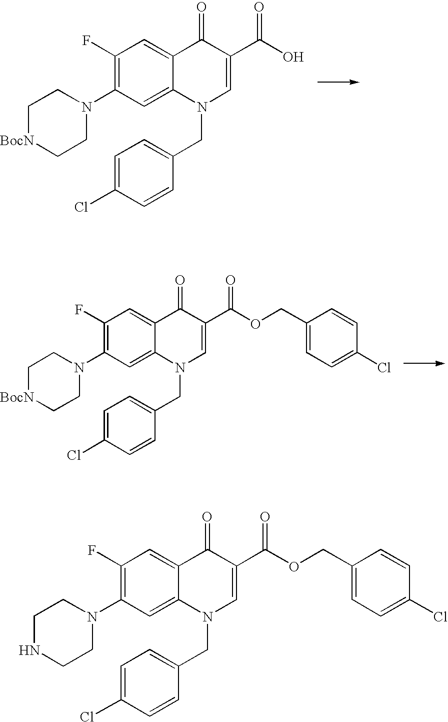
Step 6:

To a stirred solution of 7-(4-tert-butoxycarbonylpiperazin-1-yl)-1-(4-chlorobenzyl)-6-fluoro-4-oxo-1,4-dihydroquinoline-3-carboxylic acid (250 mg, 0.48 mmols) in DMF (7 mL) was added potassium carbonate (134 mg, 0.97 mmols) and 1.5 eq of 4-chloro-benzyl bromide (148 mg, 0.72 mmols) and stirred at 60° C. for 2 hr. Once the reaction was complete, reaction mixture was filtered and solvent was evaporated. 4 M HCl in dioxane was added to the residue and stirred at room temp for 2 hr at room temperature. The solvent was evaporated and residue was purified by reverse phase HPLC to give the title compound. m/z: 540 (M+1).

### Example 2

### Synthesis of 1-(4-methanesulfonylbenzyl)-3-(4-methylbenzoyl)-4-oxo-1,4-dihydroquinoline-7-carbonitrile and 1-(4-methanesulfonylbenzyl)-3-(4-methylbenzoyl)-4-oxo-1,4-dihydro-quinoline-5-carbonitrile

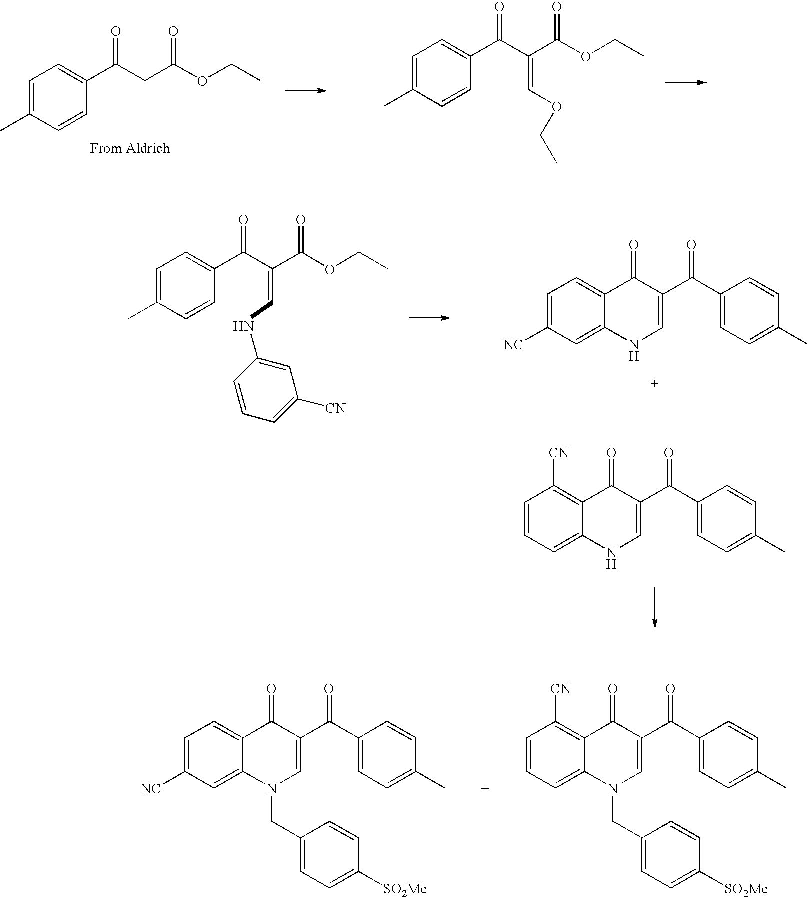 Step 1

3-Oxo-3-p-tolylpropionic acid ethyl ester (1.4 gm, 6.7 mmols), triethylorthoformate (1.82 mL, 10.9 mmols) and acetic anhydride (1.6 mL, 16.7 mmols) were heated together at reflux at −160° C. for 4 hrs. After reaction was complete, the reaction mixture was concentrated on the rotovap and dried over high vacuum to give 3-ethoxy-2-(4-methylbenzoyl)acrylic acid ethyl ester which was used as such in the next step without further purification.

Step 2

To a magnetically stirred solution of 3-ethoxy-2-(4-methylbenzoyl)acrylic acid ethyl ester (3.2 gm (crude), 10.9 mmol) in isopropanol (60 mL) was added 3-aminobenzonitrile (1.3 gm, 10.9 mmol) and and the reaction mixture was refluxed for 6 hr. After the reaction was complete, solvent was evaporated and the residue was purified with ethylacetate/hexane on a CombiFlash chromatographic system to give 3-(3-cyanophenylamino)-2-(4-methylbenzoyl)acrylic acid ethyl ester as a solid.

Step 3

To refluxing diphenyl ether (70 mL) was slowly added 3-(3-cyanophenylamino)-2-(4-methylbenzoyl)acrylic acid ethyl ester (835 mg, 2.5 mmol) (either solid or solution in diphenyl ether) and the solution was refluxed for 60 min. After the reaction was complete the reaction mixture was slowly cooled to room temperature where upon 3-(4-methylbenzoyl)-4-oxo-1,4-dihydro-quinoline-7-carbonitrile and 3-(4-methylbenzoyl)-4-oxo-1,4-dihydro-quinoline-5-carbonitrile crystallized out.

Step 4

To a stirred solution of 3-(4-methylbenzoyl)-4-oxo-1,4-dihydro-quinoline-7-carbonitrile and 3-(4-methylbenzoyl)-4-oxo-1,4-dihydro-quinoline-5-carbonitrile (200 mg, 0.69 mmols) in DMF was added potassium carbonate (191 mg, 1.4 mmols) and 1-chloromethyl-4-methanesulfonyl-benzene (commercially available from Transworld) (211 mg, 1.0 mmols) and the reaction mixture was heated at 60° C. for 2 hrs. After the reaction was complete, solvent was evaporated and the residue was purified on a reverse phase HPLC to separate the title compounds as off white solids. LCMS m/z. (M+1) 457.

### Example 3

### Synthesis of 1-(4-chlorobenzyl)-6-fluoro-7-morpholin-4-yl-4-oxo-1,4-dihydroquinoline-3-carboxylic acid 4-chlorobenzylamide

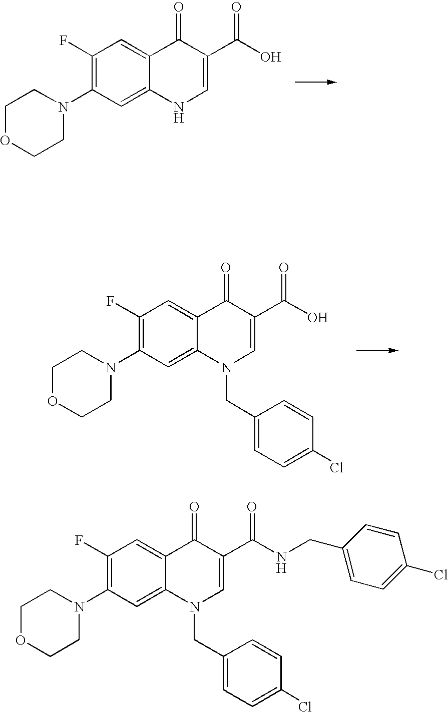 Step 1

To magnetically stirred solution of 6-fluoro-7-morpholin-4-yl-4-oxo-1,4-dihydro-quinoline-3-carboxylic acid (500 mg, 1.7 mmols) in DMF (7 mL) was added solid potassium carbonate (469 mg, 3.4 mmols) and 4-chlorobenzyl bromide (888 mg, 4.33 mmols) and the reaction mixture was stirred at 60° C. for 2 hr. After the reaction was complete, solid potassium carbonate was filtered and the solvent was evaporated. The residue was redissolved in MeOH and 50% aq. NaOH (5 mL each) and refluxed for 2 hrs. The reaction mixture was acidified and 1-(4-chlorobenzyl)-6-fluoro-7-morpholin-4-yl-4-oxo-1,4-dihydroquinoline-3-carboxylic acid, which crashed out of the solution, was filtered and dried and it was used as such in the next step.

Step 2

To a magnetically stirred solution of 1-(4-chlorobenzyl)-6-fluoro-7-morpholin-4-yl-4-oxo-1,4-dihydroquinoline-3-carboxylic acid (500 mg, 1.2 mmols) in DMF (7 mL) was added HATU (912 mg, 2.4 mmol) and 2,4,6-collidine (806 mg, 6 mmol) and the reaction mixture was stirred at room temp for 40 min. 4-Chlorobenzylamine (253 mg, 1.8 mmols) was added to the reaction mixture and allowed to stir overnight. Solvent was evaporated and residue was purified on the reverse phase HPLC to give 1-(4-chlorobenzyl)-6-fluoro-7-morpholin-4-yl-4-oxo-1,4-dihydro-quinoline-3-carboxylic acid 4-chloro-benzylamide. LCMS: m/z. (M+1) 540.

### Example 4

### Synthesis of 6,7-dimethoxy-3-(piperidine-1-carbonyl)-1-(4-trifluoromethylbenzyl)-1H-quinolin-4-one

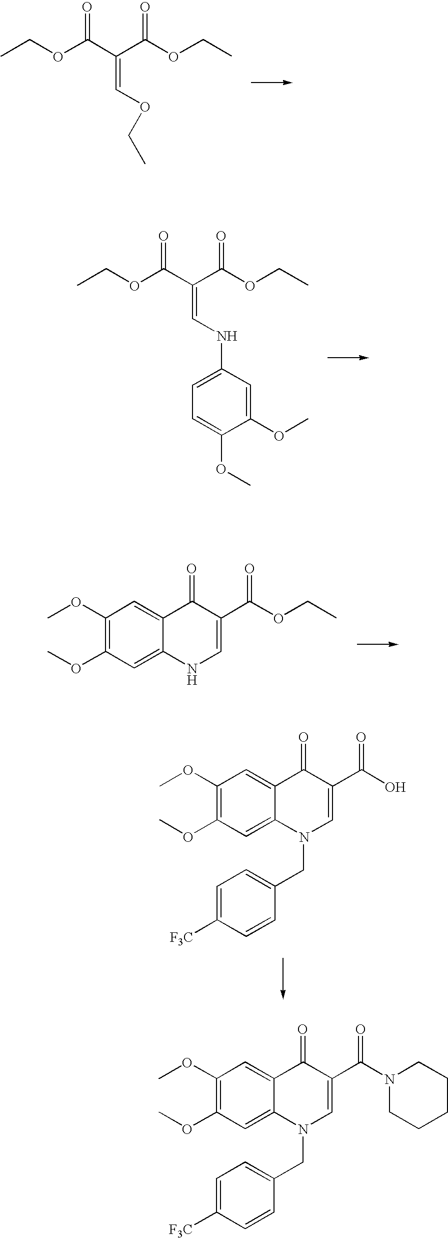 Step 1

2-[(3,4-Dimethoxy-phenylamino)-methylene]-malonic acid diethyl ester was prepared as described in Example 2, Step 2 above by reacting 2-ethoxymethylenemalonic acid diethyl ester (5 gm, 4.62 mmols) with 3,4-dimethoxyaniline (708 mg, 4.62 mmols)

Step 2

6,7-Dimethoxy-4-oxo-1,4-dihydro-quinoline-3-carboxylic acid ethyl ester was prepared by proceeding as described in Example 2, Step 3 above using 2-[(3,4-dimethoxyphenylamino)-methylene]-malonic acid diethyl ester in diphenyl ether.

Step 3

To a stirred solution of 6,7-Dimethoxy-4-oxo-1,4-dihydro-quinoline-3-carboxylic acid ethyl ester (250 mg, 0.9 mmols) in DMF (10 mL) was added potassium carbonate (248 mg, 1.8 mmols) and 4-trifluoromethylbenzyl bromide (322 mg, 1.35 mmols) and heated at 60° C. for 2 hrs. After the reaction was complete, solid potassium carbonate from the reaction was filtered and the solvent was evaporated on the rotovap. To this residue was added 50% aq. NaOH and MeOH (5 mL each) and heated to reflux for 3 hrs. The reaction mixture was cooled to room temperature and the white solid that precipitated was filtered. The solid was stirred in 4 M HCl for 10 min and filtered to give 6,7-dimethoxy-4-oxo-1-(4-trifluoromethylbenzyl)-1,4-dihydroquinoline-3-carboxylic acid which was dried and converted to the title compound using the method described in Example 3, Step 2 above. LCMS m/z 475.

### Example 5

### Synthesis of 1-(2-Fluoro-4-trifluoromethyl-benzyl)-6,7-dimethoxy-3-(4-methylbenzoyl)-1H-quinolin-4-one

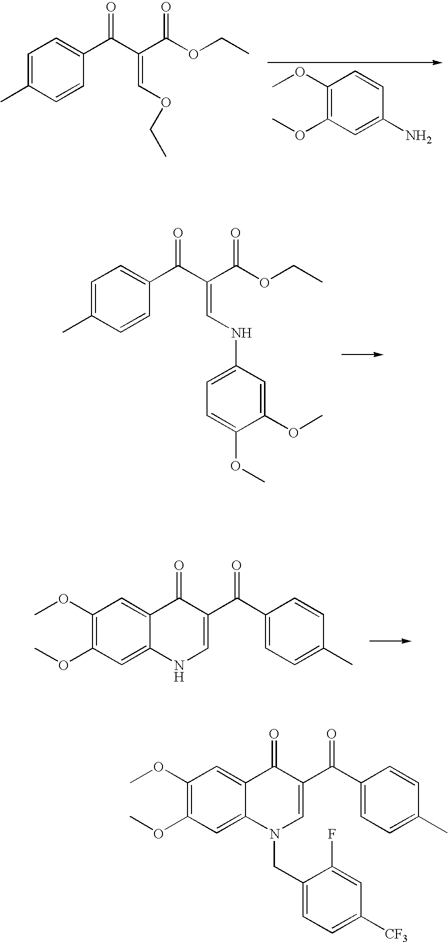

6,7-Dimethoxy-3-(4-methyl-benzoyl)-1H-quinolin-4-one was prepared by reacting 3-ethoxy-2-(4-methylbenzoyl)-acrylic acid ethyl ester with 3,4-dimethoxyaniline as described in Example 2, Steps 2, 3 and 4 above and was converted to the title compound by reacting 6,7-dimethoxy-3-(4-methyl-benzoyl)-1H-quinolin-4-one (200 mg, 0.62 mmols) with 2-fluoro-4-(trifluoromethyl)benzyl bromide (commercially available from Avocado) (239 mg, 0.93 mmols) by proceeding as described in Example 2, Step 4 above in 70% yield. LCMS m/z (M+1) 500.

### Example 6

### Synthesis of 3-(6-dimethylaminopyridine-3-carbonyl)-1-(4-methanesulfonylbenzyl)-6,7-dimethoxy-1H-quinolin-4-one

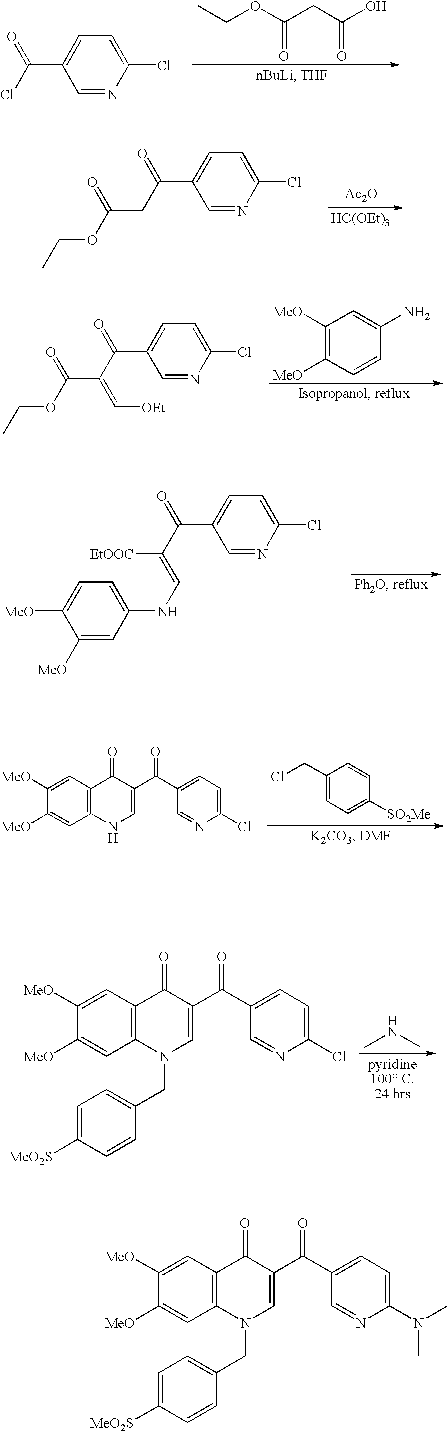 Step 1

Ethyl malonate (5 g, 37.8 mmol) in THF (106 mL) was cooled to −78° C. under nitrogen atmosphere. n-BuLi (30.2 mL, 2.5 M solution in THF, 75.6 mmol) was added via syringe over 10 min and stirring was continued at −78° C. 6-Chloronicotinoyl chloride (2.2 g, 12.6 mmol) was added dropwise over 5 min and stirring was continued at −78° C. for an additional hour. The cooling bath was then removed and after 1 hr, ether (80 mL) and 1N HCl (70 mL) were added to the reaction mixture. The organic layer was separated and washed with saturated NaHCO₃, water and brine, dried over MgSO₄ and concentrated. The resulting solid was further dried under high vacuum to afford 2.9 g of 3-(6-chloropyridin-3-yl)-3-oxopropionic acid ethyl ester as an orange brown solid.

Step 2

3-(6-Chloropyridin-3-yl)-3-oxopropionic acid ethyl ester was converted to 3-(6-chloro-pyridine-3-carbonyl)-1-(4-methanesulfonyl-benzyl)-6,7-dimethoxy-1H-quinolin-4-one by proceeding as described in Example 2, Step 1-4 above but substituting 3-aminobenzonitrile with 3,4-dimethoxyaniline.

Step 3

3-(6-Chloropyridine-3-carbonyl)-1-(4-methanesulfonylbenzyl)-6,7-dimethoxy-1H-quinolin-4-one (0.025 g, 0.05 mmol) was placed in a tube and pyridine (0.3 mL) and dimethyl amine (0.03 mL, 2M solution in THF, 0.06 mmol) were added and the tube sealed and heated at 100° C. for 48 hrs. The reaction mixture was added to water to give the title compound as a brown solid (0.01 gm, 38%). LCMS M+! 522.3, M+23 544.3, M−1 520.5.

### Example 7

### Synthesis of N-[1-(4-chlorobenzyl)-6-fluoro-7-(4-methylpiperazin-1-yl)-4-oxo-1,4-dihydro-quinolin-3-yl]-2-phenylacetamide

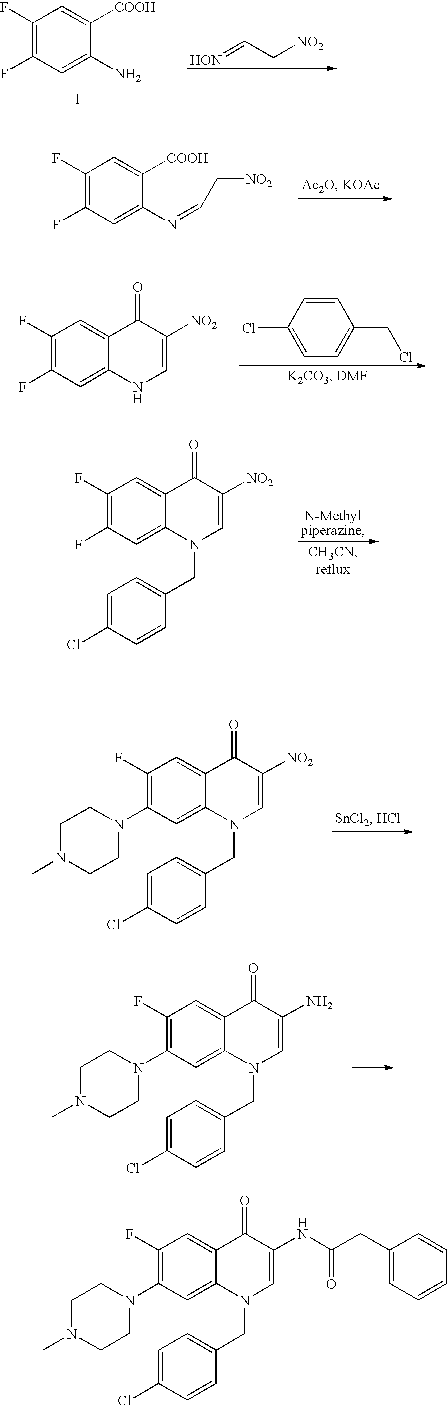 Step 1

1-(4-Chlorobenzyl)-6,7-difluoro-3-nitro-1H-quinolin-4-one was synthesized from 4,5-difluoroanthranilic acid using procedures as described in Krishnan, R.; Lang, Jr., S. A.; Siegel, M. M., J. Het. Chem. 23, 1801, 1986; and Bachman, G. B.; Welton, D. E.; Jenkins, G. L.; Christian, J. E., J. Am. Chem. Soc. 69, 365, 1947.

Step 2

1-(4-Chlorobenzyl)-6,7-difluoro-3-nitro-1H-quinolin-4-one (0.57 g, 1.6 mmol) was dissolved in acetonitrile (15 mL) and N-methylpiperazine (0.36 mL, 3.2 mmol) was added. The reaction mixture was refluxed for 4 h. The resulting crude solution of product was concentrated, then suspended in water. The solids were filtered and washed with water and dried to afford 1-(4-chlorobenzyl)-6-fluoro-7-(4-methylpiperazin-1-yl)-3-nitro-1H-quinolin-4-one (0.6 gm, 81).

Step 3

1-(4-Chlorobenzyl)-6-fluoro-7-(4-methyl-piperazin-1-yl)-3-nitro-1H-quinolin-4-one (0.69 g, 1.6 mmol) was suspended in a solution of SnCl₂.2H₂O (1.1 g, 4.8 mmol) in concentrated HCl (11 mL) at 95° C. The reaction mixture was heated at 95° C. for 3 h, then allowed to cooled to room temp. The solids were filtered, washed with water and dried on high vacuum to afford 3-amino-1-(4-chlorobenzyl)-6-fluoro-7-(4-methylpiperazin-1-yl)-1H-quinolin-4-one (0.4 gm, 62%, assumed as the dihydrochloride salt) as a yellow solid.

Step 4

3-Amino-1-(4-chloro-benzyl)-6-fluoro-7-(4-methyl-piperazin-1-yl)-1H-quinolin-4-one (0.078 g, 0.165 mmol) was dissolved in 2.5 mL of DCM and triethylamine (0.07 mL, 0.66 mmol). Phenylacetyl chloride (0.024 mL, 0.18 mmol) was added and the mixture stirred at room temperature overnight. Water (5 mL) was added to the reaction mixture and the solids that crashed out were filtered and dried on high vacuum to afford N-[1-(4-chlorobenzyl)-6-fluoro-7-(4-methylpiperazin-1-yl)-4-oxo-1,4-dihydro-quinolin-3-yl]-2-phenylacetamide (0.03 g, 35%) as a light brown solid. LCMS Calcd. mw 518.2, Observed M+1 519.0.

### Example 8

### Synthesis of N-[1-(4-Chloro-benzyl)-6-fluoro-7-(4-methyl-piperazin-1-yl)-4-oxo-1,4-dihydro-quinolin-3-yl]-C-phenyl-methanesulfonamide

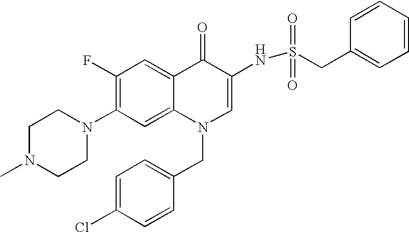

3-Amino-1-(4-chloro-benzyl)-6-fluoro-7-(4-methyl-piperazin-1-yl)-1H-quinolin-4-one (0.042 g, 0.09 mmol) was dissolved in 1.5 mL of DCM and triethylamine (0.05 mL, 0.36 mmol). Benzylsulfonyl chloride (0.017 g, 0.09 mmol) was added and the mixture stirred at room temperature overnight. Water (5 mL) was added to the reaction mixture and the solids that crashed out were filtered and subjected to purification by reverse phase HPLC followed by drying by lyophilization to afford the title compound (0.002 g, 4%) as an off-white solid. LCMS Calcd. mw 555.07, Observed M+1 556, M−1 553.5.

### Example 9

### Synthesis of 1-[1-(4-chlorobenzyl)-6-fluoro-7-(4-methylpiperazin-1-yl)-4-oxo-1,4-dihydro-quinolin-3-yl]-3-(4-chlorophenyl)urea

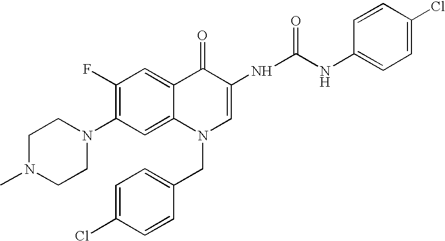

3-Amino-1-(4-chloro-benzyl)-6-fluoro-7-(4-methyl-piperazin-1-yl)-1H-quinolin-4-one (0.044 g, 0.09 mmol) was dissolved in 1.5 mL of DMF and triethylamine (0.05 mL, 0.36 mmol). 4-Chlorophenylisocyanate (0.014 g, 0.09 mmol) was added and the mixture stirred at room temperature overnight. Water (5 mL) was added to the reaction mixture and the solids that crashed out were filtered and subjected to purification by reverse phase HPLC followed by drying by lyophilization to afford the title compound (0.01 gms, 20%) as a yellow solid. LCMS Calcd. mw 554.45, Observed M+1 555.3, M−1 552.4.

### BIOLOGICAL EXAMPLES

### Example 1

### HCV Replicon Assay

The HCV replicon assay is a cell-culture system that mimics in vivo HCV replication and provides a system to study HCV replication in vitro. It was created by transfecting cloned viral RNA derived from a consensus HCV genomic sequence into human Huh7 hepatoma cells that are semi-permissive for viral RNA production (Lohmann V., Korner F., Koch J.-O., Herian U., Theilmann L. and Bartenschlager R. (1999). Replication of subgenomic Hepatitis C virus RNAs in a hepatoma cell line. Science 285, 110-113 and Blight K. J., Kolykhalov A. A. and Rice C. M. (2000). Efficient initiation of HCV RNA Replication in cell culture. Science 290, 972-1974). These transfected cell lines contain a subgenomic HCV RNA genome that includes (1) the HCV 5′NTR fused to 12 amino acids of the capsid coding region, (2) the neomycin phosphotransferase gene (Neo) as a selectable marker, (3) the internal ribosome entry site (IRES) from encephalomyocarditis virus (EMCV) that directs translation of HCV non-structural proteins (variously NS2 or NS3 to NS5B), and (4) the 3′ NTR. Replicon-containing cells autonomously and persistently replicate HCV RNA that can be measured quantitatively by real-time qPCR. Therefore, the replicon system facilitates quantitative assessment of anti-viral activity by monitoring changes in HCV RNA replication in a cell-based assay.

HCV replicon-containing cells (Huh7/Clone A) were routinely maintained in Clone A growth medium (DMEM medium [Invitrogen], supplemented with 10% Fetal Bovine Serum, 1% Non Essential Amino Acids and 1 g/L G418). Test compounds were dissolved in dimethyl sulfoxide (DMSO) to make 200×stock solutions for all doses prior to treatment.

For the HCV replicon assay, Huh7/Clone A cells were trypsinized from culture flasks, seeded in 1 ml of Clone A growth medium without G418 at 4×10⁴ cells per well in 24-well plates and incubated at 37° C. in a humidified CO₂ (5%) incubator overnight. Following overnight incubation, compound solutions were added into wells in the same volume (5 μl of 200×compound stock per well) to give a final DMSO concentration of 0.5%. Three wells on each plate supplemented with 5 μl of DMSO served as untreated controls. For IC₅₀ determinations, compounds were tested at 7 serial dilutions in triplicates from the starting stock solutions. The plates were incubated at 37° C. for 48 hours. After incubation, cells were harvested, transferred to 96-well plates, and subjected to total RNA extraction using the RNA Isolation Kit (RNeasy 96, Qiagen) according to the protocol described by the manufacture's RNeasy 96 Handbook (Qiagen).

Total RNA eluted in 130 μl of RNase-freed H₂O was quantitated by the RiboGreen Assay according to the supplier's protocol (Molecular Probe). Briefly, 5 μl of RNA samples were aliquoted in duplicate to a 96-well black microplate and a 96-well TaqMan Optical plate. RNA samples in the black microplate were mixed with 95 μl of diluted RiboGreen reagent (1:250 dilution in TE buffer) and sample fluorescence was measured using a fluorescence microplate reader at standard fluorescein wavelengths (excitation ˜480 nm, emission ˜520 nm). Ribosomal RNA (Molecular Probe) was used as standard.

TaqMan quantitative PCR (RT-qPCR) was used to quantitate the amount of HCV replicon RNA in each sample. The RT-qPCR reactions were performed in 25 μl on an ABI PRISM 7900 HT Sequence Detection System (Applied Biosystems). The reaction mixture contained 5 μl of total RNA (10-100 ng), 1×TaqMan Buffer A (Applied Biosystems), 5.5 mM MgCl₂, 1.2 mM dNTP mix, 0.625 U of AmpliTaq Gold (Applied Biosystems), 5 U of MMLV reverse transcriptase (Promega), 5 U of rRNasin (Promega), 300 nM each of the forward and reverse primers, and 100 nM TaqMan MGB probe. Primers and probe were designed to hybridize to a portion of the neomycin resistance gene (neo) in the replicon and the sequences are as follows: forward primer 5′-GGCTACCTGCCCATTCGA-3′; reverse primer 5′-CCGGCTTCCATCCGAGTAC-3′; MGB probe 5′-CCACCAAGCGAAACA-3′. The RT step was performed at 48° C. for 30 min, followed by 10 min at 95° C. The thermal cycling program consisted of 40 cycles of 15 s at 95° C. and 1 min at 60° C. TaqMan raw data (Ct values) were analyzed using the Sequence Detection System (SDS) software, mathematically converted to HCV RNA genome amount and normalized to total RNA in each sample. The sample without compound treatment served as a control and the HCV replicon RNA level from untreated cells was defined as 100%. Compound inhibitory activity was determined as the ratio of the normalized HCV RNA amount in treated samples relative to the untreated control. Compound IC₅₀s were calculated using a standard 4 parameter curve fit model.

Compounds of the invention were tested by the above-described assay and observed to inhibit HCV replication. Compounds 1, 3, 52, 53, 54, 55 and 59 inhibited HCV replication with IC50's <5 micromolar while compounds 56 and 64 inhibited HCV replication with IC50's <50 micromolar.

### Example 1

### Representative Pharmaceutical Formulations Containing a Compound of Formula (I)

ORAL FORMULATION

| | | |
|---|---|---|
| Compound of Formula (I) | 10-100 | mg |
| Citric Acid Monohydrate | 105 | mg |
| Sodium Hydroxide | 18 | mg |
| Flavoring | | |

| | |
|---|---|
| Water | q.s. to 100 mL |

INTRAVENOUS FORMULATION

| Compound of Formula (I) | 0.1-10 | mg |
|---|---|---|
| Dextrose Monohydrate | q.s. to make isotonic | |
| Citric Acid Monohydrate | 1.05 | mg |
| Sodium Hydroxide | 0.18 | mg |

| | |
|---|---|
| Water for Injection | q.s. to 1.0 mL |

TABLET FORMULATION

| | |
|---|---|
| Compound of Formula (I) | 1% |
| Microcrystalline Cellulose | 73% |
| Stearic Acid | 25% |
| Colloidal Silica | 1% |

The foregoing invention has been described in some detail by way of illustration and example, for purposes of clarity and understanding. It will be obvious to one of skill in the art that changes and modifications may be practiced within the scope of the appended claims. Therefore, it is to be understood that the above description is intended to be illustrative and not restrictive. The scope of the invention should, therefore, be determined not with reference to the above description, but should instead be determined with reference to the following appended claims, along with the full scope of equivalents to which such claims are entitled.

What is claimed is:

1. A compound of Formula Formula (I): 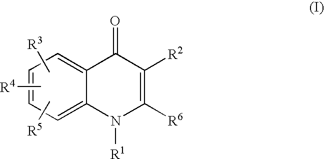
where:
R¹ is -(alkylene)-R⁷ where the alkylene chain is optionally substituted with one or two halo and R⁷ is cycloalkyl, aryl, heteroaryl, or heterocycloalkyl wherein the aromatic and alicyclic rings are optionally substituted with one, two, or three R^{a} independently selected from alkyl, alkoxy, hydroxyl, halo, haloalkyl, haloalkoxy, alkylsulfonyl, hydroxyalkyl, alkoxyalkyl, aminoalkyl, alkylamino, hydroxyalkoxy, alkoxyalkyloxy, aminoalkoxy, optionally substituted phenyloxy, optionally substituted phenalkyloxy, optionally substituted heteroaryloxy, optionally substituted heteroaralkyloxy, optionally substituted heterocycloalkyloxy, optionally substituted heterocycloalkylalkyloxy, carboxy, carboxyalkyl, alkoxycarbonyl, acyl, acylamino, acylaminoalkyl, acyloxy, aminocarbonyl, aminocarbonylalkyl, aminosulfonyl, aminosulfonylalkyl, sulfonylamino, sulfonylaminoalkyl, cyano, ureido, ureidoalkyl, aminocarbonyloxy, aminocarbonylalkyloxy, or alkoxycarbonylamino;
R² is —COR⁸ (where R⁸ is cycloalkyl, cycloalkylalkyl, aryl, aralkyl, heteroaryl, or heteroaralkyl), —COOR⁹ (where R⁹ is alkyl, cycloalkyl, cycloalkylalkyl, heterocycloalkylalkyl, aryl, aralkyl, heteroaryl, or heteroaralkyl), —CONR¹⁰R¹¹ (where R¹⁰ hydrogen, alkyl, aryl, aralkyl, heteroaryl, or heteroaralkyl and R¹¹ is alkyl, cycloalkyl, cycloalkylalkyl, heterocycloalkylalkyl, aryl, aralkyl, heteroaryl, or heteroaralkyl or R¹⁰ and R¹¹ together with the nitrogen atom to which they are attached form heterocycloamino), —NR¹²R¹³ (where R¹² is hydrogen, alkyl, hydroxyalkyl, alkoxyalkyl, aryl, aralkyl, heteroaryl, heteroaralkyl, or heterocycloalkylalkyl and R¹³ is hydrogen, alkyl, cycloalkyl, cycloalkylalkyl, aryl, aralkyl, heteroaryl, heteroaralkyl, or heterocycloalkylalkyl), —C(OH)R¹⁴R⁸ (where R¹⁴ is hydrogen, alkyl, aryl, aralkyl, heteroaryl, or heteroaralkyl and R⁸ is aryl, aralkyl, heteroaryl, or heteroaralkyl), —SO₂NR¹⁶R¹⁷ (where R¹⁶ hydrogen, alkyl, aryl, aralkyl, heteroaryl, or heteroaralkyl and R¹⁷ is alkyl, aryl, aralkyl, heteroaryl, or heteroaralkyl or R¹⁶ and R¹⁷ together with the nitrogen atom to which they are attached form heterocycloamino), —NHSO₂R¹⁸ (where R¹⁸ is aryl, aralkyl, heteroaryl, heteroaralkyl, or heterocycloalkyl), —NHCOOR¹⁹ (where R¹⁹ is aryl, aralkyl, heteroaryl, heteroaralkyl, or heterocycloalkyl), —NHCONHR²⁰R²¹ (where R²⁰ is hydrogen or alkyl and R²¹ is aryl, aralkyl, heteroaryl, heteroaralkyl, or heterocycloalkyl or R²⁰ and R²¹ together with the nitrogen atom to which they are attached form heterocycloamino), —SR²², —SOR²² (where each R²² is aryl, aralkyl, heteroaryl, or heteroaralkyl); —SO₂R²³ (where R²³ is aryl, aralkyl, heteroaryl, or heteroaralkyl), or —SO₃R²⁴ where R²⁴ is aryl, aralkyl, heteroaryl, or heteroaralkyl); wherein the aromatic, alicyclic or heterocycloalkyl ring in R² is optionally substituted with one, two, or three R^{b} independently selected from alkyl, alkoxy, hydroxyl, halo, haloalkyl, haloalkoxy, alkylsulfonyl, hydroxyalkyl, alkoxyalkyl, aminoalkyl, alkylamino, hydroxyalkoxy, alkoxyalkyloxy, aminoalkoxy, optionally substituted phenyloxy, optionally substituted phenylalkyloxy, optionally substituted heteroaralkyloxy, optionally substituted heterocycloalkyloxy, carboxy, carboxyalkyl, alkoxycarbonyl, alkoxycarbonylalkyl, acyl, acylamino, acylaminoalkyl, acyloxy, aminocarbonyl, aminocarbonylalkyl, aminosulfonyl, aminosulfonylalkyl, sulfonylamino, sulfonylaminoalkyl, cyano, ureido, ureidoalkyl, aminocarbonyloxy, or alkoxycarbonylamino;
R³ is hydrogen, alkyl, halo, alkoxy, hydroxy, haloalkoxy, or haloalkyl;
R⁴ is hydrogen, alkyl, halo, alkoxy, hydroxy, haloalkoxy, haloalkyl, alkylthio, alkylsulfonyl, hydroxyalkyl, alkoxyalkyl, aminoalkyl, hydroxyalkoxy, alkoxyalkyloxy, aminoalkoxy, aryloxy, aralkyloxy, heteroaralkyloxy, heterocycloalkyloxy, heterocycloalkylalkyloxy, carboxyalkyl, alkoxycarbonylalkyl, carboxy, alkoxycarbonyl, acyl, acylamino, acyloxy, aminocarbonyl, aminocarbonylalkyloxy, aminosulfonyl, cyano, ureido, aminocarbonyloxy, or alkoxycarbonylamino wherein the aromatic or alicyclic ring in aryloxy, aralkyloxy, heteroaralkyloxy, heterocycloalkyloxy, and heterocycloalkylalkyloxy is optionally substituted with one, two, or three R^{c} independently selected from alkyl, alkoxy, hydroxyl, halo, haloalkyl, haloalkoxy, alkylsulfonyl, hydroxyalkyl, alkoxyalkyl, aminoalkyl, hydroxyalkoxy, alkoxyalkyloxy, aminoalkoxy, optionally substituted phenyloxy, optionally substituted phenylalkyloxy, optionally substituted heteroaryloxy, optionally substituted heteroaralkyloxy, optionally substituted heterocycloalkyloxy, optionally substituted heterocycloalkylalkyloxy, carboxy, carboxyalkyl, alkoxycarbonyl, acyl, acylamino, acylaminoalkyl, acyloxy, aminocarbonyl, aminocarbonylalkyl, aminosulfonyl, aminosulfonylalkyl, sulfonylamino, sulfonylaminoalkyl, cyano, ureido, ureidoalkyl, aminocarbonyloxy, aminocarbonylalkyloxy, or alkoxycarbonylamino; and
R⁵ is hydrogen, alkyl, halo, alkoxy, hydroxy, haloalkoxy, haloalkyl, aryl, aralkyl, heteroaryl, heteroaralkyl, cycloalkyl, heterocycloalkyl, heterocycloalkylalkyl, heterocycloalkylalkyloxy, monosubstituted amino, or disubstituted amino or when R⁵ together with R³ or R⁴ forms methylenedioxy or ethylenedioxy wherein the aromatic and alicyclic ring in aryl, aralkyl, heteroaryl, heteroaralkyl, heterocycloalkyl, heterocycloalkylalkyl, and heterocycloalkylalkyloxy is optionally substituted with one, two, or three R^{d} independently selected from alkyl, alkoxy, hydroxyl, halo, haloalkyl, haloalkoxy, alkylsulfonyl, hydroxyalkyl, alkoxyalkyl, aminoalkyl, hydroxyalkoxy, alkoxyalkyloxy, aminoalkoxy, optionally substituted phenyloxy, optionally substituted phenylalkyloxy, optionally substituted heteroaryloxy, optionally substituted heteroaralkyloxy, optionally substituted heterocycloalkyloxy, optionally substituted heterocycloalkylalkyloxy, carboxy, carboxyalkyl, alkoxycarbonyl, acyl, acylamino, acylaminoalkyl, acyloxy, aminocarbonyl, aminocarbonylalkyl, aminosulfonyl, aminosulfonylalkyl, sulfonylamino, sulfonylaminoalkyl, cyano, ureido, ureidoalkyl, aminocarbonyloxy, aminocarbonylalkyloxy, or alkoxycarbonylamino; and
R⁶ is hydrogen, alkyl, halo, alkoxy, alkylamino, dialkylamino, phenyl, heteroaryl, carboxy, alkoxycarbonyl, or optionally substituted heterocycloalkylalkyloxycarbonyl; or
a pharmaceutically acceptable salts thereof, provided that the compound of Formula (I) is not:
L-proline, 1-[[1,4-dihydro-4-oxo-1-(phenylmethyl)-3-quinolinyl]carbonyl] ethyl ester;
3-benzoyl-6-methyl-1-(phenylmethyl)-4(1H)quinolinone;
6-methoxy-3-(4-methoxybenzoyl)-1-(phenylmethyl)-4(1H)quinolinone;
3-benzoyl-5,7-dimethoxy-1-(phenylmethyl)-4(1H)quinolinone;
3-(4-methoxybenzoyl)-6-methyl-1-(phenylmethyl)-4(1H)quinolinone;
3-(1,3-benzodioxol-5-ylcarbonyl)-1-(phenylmethyl)-4(1H)quinolinone;
1-[(1,4-dihydro-4-oxo-1-phenethyl-3-quinolyl)carbonyl]piperidine;
1-[(1,4-dihydro-4-oxo-1-phenethyl-3-quinolyl)carbonyl]-4-methylpiperazine;
1-[(1,4-dihydro-4-oxo-1-phenethyl-3-quinolyl)carbonyl]morpholine; and
when R¹ is benzyl optionally substituted with methyl, chloro, methoxy, or fluoro, R² is —COR⁸ where R⁸ is phenyl optionally substituted with methyl, ethyl, methoxy, ethoxy, fluoro, or chloro or phenyl disubstituted with methyl, R³ is hydrogen, and R⁴ is methyl, methoxy, ethoxy, or fluoro, then R⁵ is not hydrogen, methoxy, or fluoro.

2. The compound of claim 1, wherein R¹ is -(alkylene)-R⁷ where R⁷ is aryl optionally substituted with one, two, or three R^{a}; and R⁶ is hydrogen, alkyl, or halo.

3. The compound of claim 1, wherein R¹ is benzyl or 3-phenylpropyl optionally substituted with one, two, or three R^{a} independently selected from halo, alkyl, haloalkyl, haloalkoxy, alkylsulfonyl, cyano, or alkoxycarbonyl; and R⁶ is hydrogen, alkyl, or halo.

4. The compound of claim 1, wherein R¹ is benzyl or 4-chlorobenzyl; and R⁶ is hydrogen.

5. The compound of claim 1, wherein R¹ is -(alkylene)-R⁷ where R⁷ is heteroaryl optionally substituted with one, two, or three R^{a}; and R⁶ is hydrogen, alkyl, or halo.

6. The compound of claim 1, wherein R⁴ is halo and R⁵ is heterocycloalkyl.

7. The compound of claim 1, wherein R² is —COR⁸ where R² is aryl, aralkyl, heteroaryl, or heteroaralkyl optionally substituted with one, two, or three R^{b}.

8. The compound of claim 1, wherein R² is —COOR⁹ where R⁹ is alkyl, aryl, aralkyl, heteroaryl, or heteroaralkyl optionally substituted with one, two, or three R^{b}.

9. The compound of claim 8, wherein R² is —COOR⁹ where R⁹ is aralkyl optionally substituted with one, two, or three R^{b}.

10. The compound of claim 9, wherein R² is —COOR⁹ where R⁹ is aralkyl optionally substituted with one, two, or three halo.

11. The compound of claim 1, wherein R² is —CONR¹⁰R¹¹ where R¹⁰ hydrogen, alkyl, aryl, aralkyl, heteroaryl, or heteroaralkyl and R¹¹ is alkyl, aryl, aralkyl, heteroaryl, or heteroaralkyl optionally substituted with one, two, or three R^{b}.

12. The compound of any of the claims 1-11 wherein R⁴ and R⁵ are located at C-6 and C-7 positions of the quinolone ring and R³ is located at the C-8 of the quinoline ring.

13. The compound of any of the claims 1-11 wherein R⁴ is located at the C-6 postion of the quinoline ring and is selected from halo, aminocarbonyl, aminocarbonylalkyloxy, hydroxyl, or alkoxy, and R⁵ is located at C-7 positions of the quinolone ring and is heterocycloalkyl and R³ is located at the C-8 of the quinoline ring.

14. The compound of any of the claims 1-11 wherein R⁴ is located at the C-6 position of the quinoline ring and is selected from fluoro, methoxy or —CONH₂ and R⁵ is located at C-7 positions of the quinolone ring and is piperizin-1-yl, piperidin-2-yl, morpholin-4-yl, thiomorpholin-4-yl, thiomorpholin-4-yl-S,S-dioxide or homopiperazine wherein the heterocycloalkyl ring is optionally substituted with one, two, or three R^{d} and R³ is located at the C-8 of the quinoline ring and is hydrogen or methoxy.

15. A pharmaceutical composition comprising a compound of any of the claims 1-11 in admixture with one or more suitable excipients.

16. A method for treating hepatitis C infections in an animal which method comprises administering to the animal a pharmaceutical composition comprising a therapeutically effective amount of a compound of any of the claims 1-11 in admixture with one or more suitable excipients.

17. A method in accordance with claim 16, further comprising administration of a second antiviral agent selected from the group consisting of interferon, pegylated or unpegylated congeners of interferon, Ribavirin, a HCV polymerase inhibitor and a toll receptor agonist.
